(19) United States
(12) Patent Application Publication
(10) Pub. No.: US 2020/0121509 A1
(43) Pub. Date: Apr. 23, 2020

(54) PEEL AND PLACE DRESSING HAVING A CLOSED-CELL CONTACT LAYER

(71) Applicant: KCI Licensing, Inc., San Antonio, TX (US)

(72) Inventors: Christopher Brian LOCKE, Bournemouth (GB); Timothy Mark ROBINSON, Shillingstone (GB)

(21) Appl. No.: 16/597,258

(22) Filed: Oct. 9, 2019

Related U.S. Application Data
(60) Provisional application No. 62/746,717, filed on Oct. 17, 2018.

(51) Int. Cl. A61F 13/00; A61M 1/00; A61F 13/02

(52) U.S. Cl. CPC A61F 13/00068; A61M 1/0088; A61F 13/025; A61F 2013/00795; A61F 2013/00536; A61F 2013/00604; A61F 2013/00863

(57) ABSTRACT

A dressing for treating a tissue site with negative pressure may have a first layer comprising a manifold, a second layer coupled to the first layer, a third layer coupled to the second layer opposite the first layer. The second layer is formed from a first closed-cell foam and includes a plurality of apertures through the first closed-cell foam. The third layer is formed from a second closed-cell foam and includes a plurality of fluid restrictions through the second closed-cell foam that are configured to expand in response to a pressure gradient across the second closed-cell foam. The plurality of fluid restrictions are fluidly coupled with at least some of the plurality of apertures in the second layer.

### RELATED APPLICATIONS

This application claims the benefit, under 35 U.S.C. § 119(e), of the filing of U.S. Provisional Patent Application Ser. No. 62/746,717, entitled “PEEL AND PLACE DRESSING HAVING A CLOSED-CELL CONTACT LAYER,” filed Oct. 17, 2018, which is incorporated herein by reference for all purposes.

### TECHNICAL FIELD

The invention set forth in the appended claims relates generally to tissue treatment systems and more particularly, but without limitation, to dressings for tissue treatment with negative pressure and methods of using the dressings for tissue treatment with negative pressure.

### BACKGROUND

Clinical studies and practice have shown that reducing pressure in proximity to a tissue site can augment and accelerate growth of new tissue at the tissue site. The applications of this phenomenon are numerous, but it has proven particularly advantageous for treating wounds. Regardless of the etiology of a wound, whether trauma, surgery, or another cause, proper care of the wound is important to the outcome. Treatment of wounds or other tissue with reduced pressure may be commonly referred to as “negative-pressure therapy,” but is also known by other names, including “negative-pressure wound therapy,” “reduced-pressure therapy,” “vacuum therapy,” “vacuum-assisted closure,” and “topical negative-pressure,” for example. Negative-pressure therapy may provide a number of benefits, including migration of epithelial and subcutaneous tissues, improved blood flow, and micro-deformation of tissue at a wound site. Together, these benefits can increase development of granulation tissue and reduce healing times.

There is also widespread acceptance that cleansing a tissue site can be highly beneficial for new tissue growth. For example, a wound or a cavity can be washed out with a liquid solution for therapeutic purposes. These practices are commonly referred to as “irrigation” and “lavage” respectively. “Instillation” is another practice that generally refers to a process of slowly introducing fluid to a tissue site and leaving the fluid for a prescribed period of time before removing the fluid. For example, instillation of topical treatment solutions over a wound bed can be combined with negative-pressure therapy to further promote wound healing by loosening soluble contaminants in a wound bed and removing infectious material. As a result, soluble bacterial burden can be decreased, contaminants removed, and the wound cleansed.

While the clinical benefits of negative-pressure therapy and/or instillation therapy are widely known, improvements to therapy systems, components, and processes may benefit healthcare providers and patients.

### BRIEF SUMMARY

New and useful systems, apparatuses, and methods for treating tissue in a negative-pressure therapy environment are set forth in the appended claims. Illustrative embodiments are also provided to enable a person skilled in the art to make and use the claimed subject matter.

For example, in some embodiments, a dressing for treating tissue may be a composite of dressing layers, including a first open-cell foam layer, a second closed-cell foam layer, and a third closed-cell foam layer. The first open-cell foam layer may be a manifold which is substantially open to pressure and flow. The first open-cell foam layer may be a reticulated foam and may be felted or unfelted. In some embodiments, wherein the first open-cell foam layer is felted, the first open-cell foam layer may have a thickness of about 2 millimeters to about 5 millimeters. In other embodiments, wherein the first open-cell foam layer is unfelted, the first open-cell foam layer may have a thickness of about 6 millimeters to about 10 millimeters. The second closed-cell foam layer may be bonded to the first open-cell foam layer and includes an array of apertures or holes extending through the second closed-cell foam. In some embodiments, each of the apertures may have a diameter of about 2 millimeters to about 3 millimeters. In other embodiments, each of the apertures may have a diameter greater than 3 millimeters. In some embodiments, the second closed-cell foam layer may have a thickness of about 1 millimeter to about 3 millimeters. The third closed-cell foam layer may be a tissue facing layer and may be bonded to the second closed-cell foam layer. The third closed-cell foam layer includes an array of fluid restrictions, such as fenestrations, registered with the array of apertures in the second closed-cell foam layer. In some embodiments, each of the fluid restrictions may be perforations having a length of about 2 millimeters to about 3 millimeters and a width of about 0.3 millimeters to about 0.7 millimeters. In other embodiments, each of the fluid restrictions may have a length greater than 3 millimeters and a width greater than 0.7 millimeters. In some embodiments, the third closed-cell foam layer may have a thickness of about 1 millimeter to about 3 millimeters. Both the second and third closed-cell foam layers may be hydrophobic to encourage exudate and other fluid to pass quickly from the tissue to the first open-cell layer. The second closed-cell foam layer may be less hydrophobic than the third closed-cell foam layer.

More generally, some embodiments may comprise a dressing having at least three layers in a stacked relationship. The first layer may comprise a manifold. The second layer may be coupled to the first layer and may comprise or consist essentially of a closed-cell foam having a plurality of apertures. The third layer may be coupled to the second layer opposite the first layer. The third layer may comprise or consist essentially of a closed-cell foam having a plurality of fluid restrictions. The plurality of fluid restrictions may be configured to expand in response to a pressure gradient across the second closed-cell foam. The plurality of fluid restrictions may be fluidly coupled with at least some of the plurality of apertures in the second layer.

In some embodiments, the first layer may comprise a foam, and more particularly a reticulated polymer foam that is substantially open to pressure and flow. In some examples, the foam has a free volume of at least 90%. In other examples, the foam is porous and has an average pore size in a range of about 0.4 millimeters (400 microns) to about 0.6 millimeters (600 microns). An unfelted manifold having a thickness less than about 12 millimeters may be suitable for many therapeutic applications. Additionally, a felted manifold having a thickness less than 5 millimeters may also be suitable for many therapeutic applications.

In some embodiments, the second layer may comprise a hydrophobic closed-cell foam, and more particularly a silicone, polyurethane, or ethylene vinyl acetate closed-cell foam. A second layer having a thickness in a range of about 1 millimeter to about 3 millimeters may be suitable for many therapeutic applications. In some examples, the closed-cell foam forming the second layer may have a pore size in a range of about 0.2 millimeters (200 microns) to about 1 millimeter (1000 microns) and a durometer in a range of about 10 Shore A to about 50 Shore A. In some examples, the closed-cell foam forming the second layer may have a porosity in a range of about 200 ppi (pores per inch) to about 30 ppi. In some embodiments, the second layer may be highly hydrophobic, but may be less hydrophobic than the third layer.

The apertures in the second layer may comprise a plurality of holes in some embodiments. For example, the apertures may comprise a plurality of holes having a diameter of about 10 millimeters or less. In some embodiments, the apertures may be distributed across the second layer in a uniform pattern, such as a grid of parallel rows and columns. In some embodiments, the apertures may be distributed across the second layer in parallel rows and columns, and the rows may be spaced about 20 millimeters or less apart from each other. The apertures in each of the rows may also be spaced about 10 millimeters or less apart from each other in some examples. In some embodiments, at least one of the apertures is sized to permit deflection of the second closed-cell foam proximate the plurality of fluid restrictions of about 1 millimeter into and out of the at least one aperture.

In some embodiments, the third layer may comprise a hydrophobic closed-cell foam, and more particularly a silicone, polyurethane, or ethylene vinyl acetate closed-cell foam. A third layer having a thickness in a range of about 1 millimeter to about 3 millimeters may be suitable for many therapeutic applications. In some examples, the closed-cell foam forming the third layer may have a pore size in a range of about 0.2 millimeters (200 microns) to about 1 millimeter (1000 microns) and a durometer in a range of about 10 Shore A to about 50 Shore A. In some examples, the closed-cell foam forming the third layer may have a porosity in a range of about 200 ppi to about 30 ppi. In some embodiments, the third layer may be highly hydrophobic, and may be more hydrophobic than the second layer. In some embodiments, the face of the third layer that faces the tissue site may have a smooth surface finish either due to the original manufacturing process or due to a post-formation process.

The fluid restrictions may comprise a plurality of linear slits or slots in some embodiments. For example, the fluid restrictions may comprise a plurality of linear slots having a length of approximately 5 millimeters or less, and a width of approximately 2 millimeters or less. A length of approximately 3 millimeters and a width of approximately 1 millimeter may be suitable for many therapeutic applications. In some embodiments, the fluid restrictions may be distributed across the third layer in a uniform pattern, such as a grid of parallel rows and columns. In some embodiments, the fluid restrictions may be distributed across the third layer in parallel rows and columns, and the rows may be spaced about 3 millimeters apart from each other. The fluid restrictions in each of the rows may also be spaced about 3 millimeters apart from each other in some examples. In some embodiments, the plurality of fluid restrictions has an open area in a range of about 8% to about 10% of the total area of the first layer.

In some embodiments, the fluid restrictions may be described as imperfect elastomeric valves, which may not completely close and can deform and increase in width if negative pressure is applied, providing less restriction to flow. If negative pressure is stopped or reduced, the fluid restrictions generally return to or approach their original state, providing a higher restriction to fluid flow.

In other example embodiments, the dressing may further comprise a cover coupled to the first layer opposite the second layer. Additionally, a dressing interface may be coupled to the cover, wherein the dressing interface is configured to be coupled to a fluid conductor.

In yet other example embodiments, a dressing for treating a tissue site may comprise a composite of dressing layers, including a manifold, a support layer coupled to the manifold, and a fluid control layer coupled to the support layer opposite the manifold. The support layer may comprise or consist essentially of a first closed-cell foam having a plurality of apertures. The fluid control layer may comprise or consist essentially of a second closed-cell foam having a plurality of fluid restrictions. The plurality of fluid restrictions may be configured to expand in response to a pressure gradient across the second closed-cell foam. The plurality of fluid restrictions may be fluidly coupled with at least some of the plurality of apertures in the support layer.

In yet other example embodiments, a dressing for treating a tissue site with negative pressure may comprise a first manifold layer, a second layer coupled to the first manifold layer, and a third layer coupled to the second layer opposite the first manifold layer. The second layer comprises a closed-cell foam having a plurality of apertures. The third layer comprises a closed-cell foam having a plurality of slit valves in registration with at least some of the plurality of apertures in the second layer. The plurality of slit valves are configured to be responsive to a pressure gradient.

In yet other example embodiments, a dressing for treating a tissue site with negative pressure may comprise a first manifold layer, a second layer coupled to the first manifold layer, and a third layer coupled to the second layer opposite the first layer. The second layer comprises a closed-cell hydrophobic foam and a plurality of apertures extending through the second layer. The third layer comprises a closed-cell hydrophobic foam and a plurality of fluid passages extending through the third layer, wherein the plurality of fluid passages are fluidly coupled to at least some of the plurality of apertures through the second layer. The plurality of fluid passages are configured to expand in response to a pressure gradient across the third layer.

In yet other example embodiments, a dressing for treating a tissue site with negative pressure may comprise a first manifold layer, a second layer coupled to the first manifold layer, and a third layer. The second layer comprises a first closed-cell foam. A plurality of apertures extends through the second layer. The third layer comprises a second closed-cell foam. A plurality of fluid passages extend through the third layer and are fluidly coupled to at least some of the plurality of fluid passages through the first layer, wherein the plurality of fluid passages are normally restricted and configured to expand in response to a pressure gradient across the third layer.

In yet other example embodiments, a dressing for treating a tissue site with negative pressure may comprise a cover, a manifold, a support layer comprising a first closed-cell foam having a substantially flat surface and a plurality of apertures, and a fluid control layer comprising a second closed-cell foam having a substantially flat surface and a plurality of perforations fluidly coupled with the plurality of apertures, wherein the cover, the manifold, the support layer, and the fluid control layer are assembled in a stacked relationship, and the fluid control layer is configured to contact the tissue site.

In yet other example embodiments, a dressing for treating a tissue site with negative pressure may comprise a cover, a gel layer coupled to the cover, the gel layer comprising an open central window and a plurality of openings around the open central window, a manifold, a support layer comprising a first closed-cell foam having a substantially flat surface and a plurality of apertures, and a fluid control layer comprising a second closed-cell foam having a substantially flat surface and a plurality of perforations fluidly coupled with the plurality of apertures, wherein the cover, the gel layer, the manifold, the support layer, and the fluid control layer are assembled in a stacked relationship, and the fluid control layer is configured to contact the tissue site.

In yet other example embodiments, a dressing for treating a tissue site with negative pressure may comprise a dressing for treating a tissue site with negative pressure, the dressing comprising a first layer, a second layer coupled to the manifold layer, and a third layer coupled to the second layer. The manifold layer comprises a foam having a free volume of at least 90% and a thickness in a range of about 2 millimeters to about 10 millimeters. The second layer comprises a first closed-cell hydrophobic foam having a thickness in a range of about 1 millimeter to about 3 millimeters, a pore size in a range of about 0.2 millimeters (200 microns) to about 1 millimeters (1000 microns), and a hardness of about 10 Shore A to about 50 Shore A. The dressing further includes a plurality of apertures through the first closed-cell foam comprising a plurality of holes, each of the holes having a diameter in a range of about 2 millimeters to about 3 millimeters. The third layer comprises a second closed-cell hydrophobic foam having a thickness in a range of about 1 millimeter to about 3 millimeters, a pore size in a range of about 0.2 millimeters (200 microns) to about 1 millimeters (1000 microns), and a hardness of about 10 Shore A to about 50 Shore A. The dressing further includes a plurality of fluid restrictions through the second closed-cell hydrophobic foam in registration with at least some of the plurality of apertures in the first closed-cell foam, the plurality of fluid restrictions comprising a plurality of slots configured to be responsive to a pressure gradient across the second closed-cell hydrophobic foam, each of the slots having a length in a range of about 2 millimeters to about 3 millimeters and a width in a range of about 0.3 millimeters to about 0.7 millimeters.

In yet other example embodiments, a dressing for treating a tissue site with negative pressure may comprise a first layer comprising a porous material, a second layer adjacent to the first layer, the second layer comprising a non-porous material and one or more apertures through the second layer, and a third layer adjacent to the second layer, the third layer comprising a non-porous material and one or more fluid restrictions through the third layer in registration with at least some of the one or more apertures in the second layer. The one or more fluid restrictions are configured to expand in response to a pressure gradient across the third layer.

In yet other example embodiments, a dressing for treating a tissue site with negative pressure may comprise a first layer comprising a manifold, a second layer coupled to the first layer, the second layer comprising a first closed-cell foam having an aperture through the first closed-cell foam, and a third layer comprising a second closed-cell foam having a plurality of fluid restrictions through the second closed-cell foam in registration with at least some of the plurality of apertures in the second layer. The plurality of fluid restrictions are configured to expand in response to a pressure gradient across the second closed-cell foam.

A method of treating a surface wound with negative pressure may comprise applying a dressing as described to the surface wound, sealing the dressing to epidermis adjacent to the surface wound, fluidly coupling the dressing to a source of negative-pressure, and applying negative-pressure from the negative-pressure source to the dressing. In some examples, the dressing may be applied across an edge of the surface wound, without cutting or trimming the dressing.

A method of promoting granulation in a surface wound may comprise applying a dressing to the surface wound, the dressing comprising a cover, a first layer comprising a manifold, a second layer comprising a closed-cell foam having a plurality of apertures, and a third layer comprising a closed-cell foam having a plurality of fluid restrictions fluidly coupled to the plurality of apertures. The cover may be sealed to a periwound adjacent to the surface wound, and the cover may be attached to epidermis. A negative-pressure source may be fluidly coupled to the dressing, and negative pressure from the negative-pressure source may be applied to the dressing. In some embodiments, the dressing may remain on the surface wound for at least 5 days, and at least 7 days in some embodiments. In some embodiments, a wound filler may be disposed between the third layer and the surface wound. For example, a foam wound filler may be applied to the surface wound interior to the periwound.

Objectives, advantages, and a preferred mode of making and using the claimed subject matter may be understood best by reference to the accompanying drawings in conjunction with the following detailed description of illustrative embodiments.

### BRIEF DESCRIPTION OF THE DRAWINGS

FIG. 1 is a functional block diagram of an example embodiment of a therapy system that can provide negative-pressure treatment and instillation treatment in accordance with this specification;

FIG. 2 is an exploded view of an example of a dressing, illustrating additional details that may be associated with some example embodiments of the therapy system of FIG. 1;

FIG. 3 is a schematic view of an example configuration of apertures in a layer that may be associated with some embodiments of the dressing of FIG. 2;

FIG. 4 is a schematic view of an example configuration of fluid restrictions in a layer that may be associated with some embodiments of the dressing of FIG. 2;

FIG. 5 is a schematic view of the example layer of FIG. 3 overlaid on the example layer of FIG. 4;

FIG. 6 and FIG. 7 illustrate other example configurations of fluid restrictions that may be associated with some embodiments of layers of the dressing of FIG. 2;

FIG. 8 is a flowchart illustrating a method of manufacturing layers that may be associated with some embodiments of the dressing of FIG. 2;

FIG. 9 is an exploded view of an example of a dressing, illustrating additional details that may be associated with some example embodiments of the therapy system of FIG. 1;

FIG. 10 is a top view of the example dressing of FIG. 9;

FIG. 11 is a bottom view of the example dressing of FIG. 9;

FIG. 12 is an exploded view of an example of a dressing, illustrating additional details that may be associated with some example embodiments of the therapy system of FIG. 1; and

FIG. 13 is a partial cross-section view taken along line 13-13 of a layer of the example dressing of FIG. 12.

### DESCRIPTION OF EXAMPLE EMBODIMENTS

The following description of example embodiments provides information that enables a person skilled in the art to make and use the subject matter set forth in the appended claims, but it may omit certain details already well-known in the art. The following detailed description is, therefore, to be taken as illustrative and not limiting.

The example embodiments may also be described herein with reference to spatial relationships between various elements or to the spatial orientation of various elements depicted in the attached drawings. In general, such relationships or orientation assume a frame of reference consistent with or relative to a patient in a position to receive treatment. However, as should be recognized by those skilled in the art, this frame of reference is merely a descriptive expedient rather than a strict prescription.

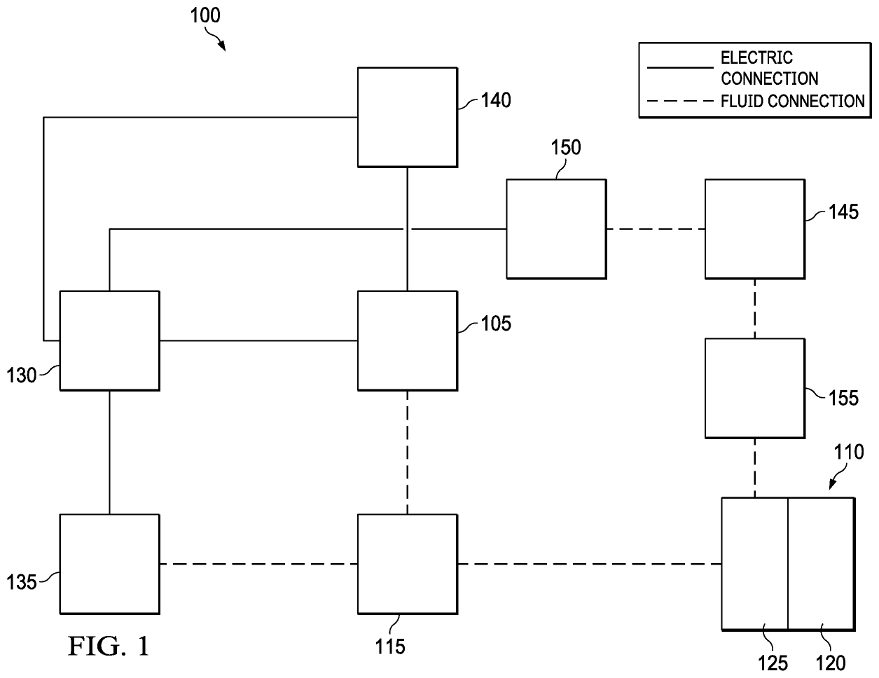

FIG. 1 is a simplified functional block diagram of an example embodiment of a therapy system 100 that can provide negative-pressure therapy with instillation of topical treatment solutions to a tissue site in accordance with this specification.

The term “tissue site” in this context broadly refers to a wound, defect, or other treatment target located on or within tissue, including, but not limited to, bone tissue, adipose tissue, muscle tissue, neural tissue, dermal tissue, vascular tissue, connective tissue, cartilage, tendons, or ligaments. A wound may include chronic, acute, traumatic, subacute, and dehisced wounds, partial-thickness burns, ulcers (such as diabetic, pressure, or venous insufficiency ulcers), flaps, and grafts, for example. The term “tissue site” may also refer to areas of any tissue that are not necessarily wounded or defective, but are instead areas in which it may be desirable to add or promote the growth of additional tissue. For example, negative pressure may be applied to a tissue site to grow additional tissue that may be harvested and transplanted.

The therapy system 100 may include a source or supply of negative pressure, such as a negative-pressure source 105, and one or more distribution components. A distribution component is preferably detachable and may be disposable, reusable, or recyclable. A dressing, such as a dressing 110, and a fluid container, such as a container 115, are examples of distribution components that may be associated with some examples of the therapy system 100. As illustrated in the example of FIG. 1, the dressing 110 may comprise or consist essentially of a tissue interface 120, a cover 125, or both in some embodiments.

A fluid conductor is another illustrative example of a distribution component. A “fluid conductor,” in this context, broadly includes a tube, pipe, hose, conduit, or other structure with one or more lumina or open pathways adapted to convey a fluid between two ends. Typically, a tube is an elongated, cylindrical structure with some flexibility, but the geometry and rigidity may vary. Moreover, some fluid conductors may be molded into or otherwise integrally combined with other components. Distribution components may also include or comprise interfaces or fluid ports to facilitate coupling and de-coupling other components. In some embodiments, for example, a dressing interface may facilitate coupling a fluid conductor to the dressing 110. For example, such a dressing interface may be a SENSAT.R.A.C.™ Pad available from Kinetic Concepts, Inc. of San Antonio, Tex.

The therapy system 100 may also include a regulator or controller, such as a controller 130. Additionally, the therapy system 100 may include sensors to measure operating parameters and provide feedback signals to the controller 130 indicative of the operating parameters. As illustrated in FIG. 1, for example, the therapy system 100 may include a first sensor 135 and a second sensor 140 coupled to the controller 130.

The therapy system 100 may also include a source of instillation solution. For example, a solution source 145 may be fluidly coupled to the dressing 110, as illustrated in the example embodiment of FIG. 1. The solution source 145 may be fluidly coupled to a positive-pressure source such as a positive-pressure source 150, a negative-pressure source such as the negative-pressure source 105, or both in some embodiments. A regulator, such as an instillation regulator 155, may also be fluidly coupled to the solution source 145 and the dressing 110 to ensure proper dosage of instillation solution (e.g. saline) to a tissue site. For example, the instillation regulator 155 may comprise a piston that can be pneumatically actuated by the negative-pressure source 105 to draw instillation solution from the solution source during a negative-pressure interval and to instill the solution to a dressing during a venting interval. Additionally or alternatively, the controller 130 may be coupled to the negative-pressure source 105, the positive-pressure source 150, or both, to control dosage of instillation solution to a tissue site. In some embodiments, the instillation regulator 155 may also be fluidly coupled to the negative-pressure source 105 through the dressing 110, as illustrated in the example of FIG. 1.

Some components of the therapy system 100 may be housed within or used in conjunction with other components, such as sensors, processing units, alarm indicators, memory, databases, software, display devices, or user interfaces that further facilitate therapy. For example, in some embodiments, the negative-pressure source 105 may be combined with the controller 130, the solution source 145, and other components into a therapy unit.

In general, components of the therapy system 100 may be coupled directly or indirectly. For example, the negative-pressure source 105 may be directly coupled to the container 115 and may be indirectly coupled to the dressing 110 through the container 115. Coupling may include fluid, mechanical, thermal, electrical, or chemical coupling (such as a chemical bond), or some combination of coupling in some contexts. For example, the negative-pressure source 105 may be electrically coupled to the controller 130 and may be fluidly coupled to one or more distribution components to provide a fluid path to a tissue site. In some embodiments, components may also be coupled by virtue of physical proximity, being integral to a single structure, or being formed from the same piece of material.

A negative-pressure supply, such as the negative-pressure source 105, may be a reservoir of air at a negative pressure or may be a manual or electrically-powered device, such as a vacuum pump, a suction pump, a wall suction port available at many healthcare facilities, or a micro-pump, for example. “Negative pressure” generally refers to a pressure less than a local ambient pressure, such as the ambient pressure in a local environment external to a sealed therapeutic environment. In many cases, the local ambient pressure may also be the atmospheric pressure at which a tissue site is located. Alternatively, the pressure may be less than a hydrostatic pressure associated with tissue at the tissue site. Unless otherwise indicated, values of pressure stated herein are gauge pressures. References to increases in negative pressure typically refer to a decrease in absolute pressure, while decreases in negative pressure typically refer to an increase in absolute pressure. While the amount and nature of negative pressure provided by the negative-pressure source 105 may vary according to therapeutic requirements, the pressure is generally a low vacuum, also commonly referred to as a rough vacuum, between −5 mm Hg (−667 Pa) and −500 mm Hg (−66.7 kPa). Common therapeutic ranges are between −50 mm Hg (−6.7 kPa) and −300 mm Hg (−39.9 kPa).

The container 115 is representative of a container, canister, pouch, or other storage component, which can be used to manage exudates and other fluids withdrawn from a tissue site. In many environments, a rigid container may be preferred or required for collecting, storing, and disposing of fluids. In other environments, fluids may be properly disposed of without rigid container storage, and a re-usable container could reduce waste and costs associated with negative-pressure therapy.

A controller, such as the controller 130, may be a microprocessor or computer programmed to operate one or more components of the therapy system 100, such as the negative-pressure source 105. In some embodiments, for example, the controller 130 may be a microcontroller, which generally comprises an integrated circuit containing a processor core and a memory programmed to directly or indirectly control one or more operating parameters of the therapy system 100. Operating parameters may include the power applied to the negative-pressure source 105, the pressure generated by the negative-pressure source 105, or the pressure distributed to the tissue interface 120, for example. The controller 130 is also preferably configured to receive one or more input signals, such as a feedback signal, and programmed to modify one or more operating parameters based on the input signals.

Sensors, such as the first sensor 135 and the second sensor 140, are generally known in the art as any apparatus operable to detect or measure a physical phenomenon or property, and generally provide a signal indicative of the phenomenon or property that is detected or measured. For example, the first sensor 135 and the second sensor 140 may be configured to measure one or more operating parameters of the therapy system 100. In some embodiments, the first sensor 135 may be a transducer configured to measure pressure in a pneumatic pathway and convert the measurement to a signal indicative of the pressure measured. In some embodiments, for example, the first sensor 135 may be a piezo-resistive strain gauge. The second sensor 140 may optionally measure operating parameters of the negative-pressure source 105, such as a voltage or current, in some embodiments. Preferably, the signals from the first sensor 135 and the second sensor 140 are suitable as an input signal to the controller 130, but some signal conditioning may be appropriate in some embodiments. For example, the signal may need to be filtered or amplified before it can be processed by the controller 130. Typically, the signal is an electrical signal, but may be represented in other forms, such as an optical signal.

The tissue interface 120 can be generally adapted to partially or fully contact a tissue site. The tissue interface 120 may take many forms, and may have many sizes, shapes, or thicknesses, depending on a variety of factors, such as the type of treatment being implemented or the nature and size of a tissue site. For example, the size and shape of the tissue interface 120 may be adapted to the contours of deep and irregular shaped tissue sites. Any or all of the surfaces of the tissue interface 120 may have an uneven, coarse, or jagged profile.

In some embodiments, the tissue interface 120 may comprise or consist essentially of a manifold. A manifold in this context may comprise or consist essentially of a means for collecting or distributing fluid across the tissue interface 120 under pressure. For example, a manifold may be adapted to receive negative pressure from a source and distribute negative pressure through multiple apertures across the tissue interface 120, which may have the effect of collecting fluid from across a tissue site and drawing the fluid toward the source. In some embodiments, the fluid path may be reversed or a secondary fluid path may be provided to facilitate delivering fluid, such as fluid from a source of instillation solution, across a tissue site.

In some embodiments, the cover 125 may provide a bacterial barrier and protection from physical trauma. The cover 125 may also be constructed from a material that can reduce evaporative losses and provide a fluid seal between two components or two environments, such as between a therapeutic environment and a local external environment. The cover 125 may comprise or consist of, for example, an elastomeric film or membrane that can provide a seal adequate to maintain a negative pressure at a tissue site for a given negative-pressure source. The cover 125 may have a high moisture-vapor transmission rate (MVTR) in some applications. For example, the MVTR may be at least 250 grams per square meter per twenty-four hours (g/m²/24 hours) in some embodiments, measured using an upright cup technique according to ASTM E96/E96M Upright Cup Method at 38° C. and 10% relative humidity (RH). In some embodiments, an MVTR up to 5,000 grams per square meter per twenty-four hours (g/m²/24 hours) may provide effective breathability and mechanical properties.

In some example embodiments, the cover 125 may be a non-porous polymer drape or film, such as a polyurethane film, that is permeable to water vapor but impermeable to liquid. Such drapes typically have a thickness in the range of 25-50 microns (μm). For permeable materials, the permeability generally should be low enough that a desired negative pressure may be maintained. The cover 125 may comprise, for example, one or more of the following materials: polyurethane (PU), such as hydrophilic polyurethane; cellulosics; hydrophilic polyamides; polyvinyl alcohol; polyvinyl pyrrolidone; hydrophilic acrylics; silicones, such as hydrophilic silicone elastomers; natural rubbers; polyisoprene; styrene butadiene rubber; chloroprene rubber; polybutadiene; nitrile rubber; butyl rubber; ethylene propylene rubber; ethylene propylene diene monomer; chlorosulfonated polyethylene; polysulfide rubber; ethylene vinyl acetate (EVA); co-polyester; and polyether block polymide copolymers. Such materials are commercially available as, for example, Tegaderm® drape, commercially available from 3M Company, Minneapolis, Minn.; polyurethane (PU) drape, commercially available from Avery Dennison Corporation, Pasadena, Calif.; polyether block polyamide copolymer (PEBAX), for example, from Arkema S. A., Colombes, France; and Inspire 2301 and Inpsire 2327 polyurethane films, commercially available from Expopack Advanced Coatings, Wrexham, United Kingdom. In some embodiments, the cover 125 may comprise INSPIRE 2301 having an MVTR (upright cup technique) of 2600 g/m²/24 hours and a thickness of about 30 microns.

An attachment device may be used to attach the cover 125 to an attachment surface, such as undamaged epidermis, a gasket, or another cover. The attachment device may take many forms. For example, an attachment device may be a medically-acceptable, pressure-sensitive adhesive configured to bond the cover 125 to epidermis around a tissue site. In some embodiments, for example, some or all of the cover 125 may be coated with an adhesive, such as an acrylic adhesive, which may have a coating weight of about 25-65 grams per square meter (g.s.m.). Thicker adhesives, or combinations of adhesives, may be applied in some embodiments to improve the seal and reduce leaks. Other example embodiments of an attachment device may include a double-sided tape, paste, hydrocolloid, hydrogel, silicone gel, or organogel.

The solution source 145 may also be representative of a container, canister, pouch, bag, or other storage component, which can provide a solution for instillation therapy. Compositions of solutions may vary according to a prescribed therapy, but examples of solutions that may be suitable for some prescriptions include hypochlorite-based solutions, silver nitrate (0.5%), sulfur-based solutions, biguanides, cationic solutions, and isotonic solutions.

In operation, the tissue interface 120 may be placed within, over, on, or otherwise proximate to a tissue site. If the tissue site is a wound, for example, the tissue interface 120 may partially or completely fill the wound, or it may be placed over the wound. The cover 125 may be placed over the tissue interface 120 and sealed to an attachment surface near a tissue site. For example, the cover 125 may be sealed to undamaged epidermis peripheral to a tissue site. Thus, the dressing 110 can provide a sealed therapeutic environment proximate to a tissue site, substantially isolated from the external environment, and the negative-pressure source 105 can reduce pressure in the sealed therapeutic environment.

The fluid mechanics of using a negative-pressure source to reduce pressure in another component or location, such as within a sealed therapeutic environment, can be mathematically complex. However, the basic principles of fluid mechanics applicable to negative-pressure therapy and instillation are generally well-known to those skilled in the art, and the process of reducing pressure may be described illustratively herein as “delivering,” “distributing,” or “generating” negative pressure, for example.

In general, exudate and other fluid flow toward lower pressure along a fluid path. Thus, the term “downstream” typically implies something in a fluid path relatively closer to a source of negative pressure or further away from a source of positive pressure. Conversely, the term “upstream” implies something relatively further away from a source of negative pressure or closer to a source of positive pressure. Similarly, it may be convenient to describe certain features in terms of fluid “inlet” or “outlet” in such a frame of reference. This orientation is generally presumed for purposes of describing various features and components herein. However, the fluid path may also be reversed in some applications, such as by substituting a positive-pressure source for a negative-pressure source, and this descriptive convention should not be construed as a limiting convention.

Negative pressure applied across the tissue site through the tissue interface 120 in the sealed therapeutic environment can induce macro-strain and micro-strain in the tissue site. Negative pressure can also remove exudate and other fluid from a tissue site, which can be collected in container 115.

In some embodiments, the controller 130 may receive and process data from one or more sensors, such as the first sensor 135. The controller 130 may also control the operation of one or more components of the therapy system 100 to manage the pressure delivered to the tissue interface 120. In some embodiments, controller 130 may include an input for receiving a desired target pressure and may be programmed for processing data relating to the setting and inputting of the target pressure to be applied to the tissue interface 120. In some example embodiments, the target pressure may be a fixed pressure value set by an operator as the target negative pressure desired for therapy at a tissue site and then provided as input to the controller 130. The target pressure may vary from tissue site to tissue site based on the type of tissue forming a tissue site, the type of injury or wound (if any), the medical condition of the patient, and the preference of the attending physician. After selecting a desired target pressure, the controller 130 can operate the negative-pressure source 105 in one or more control modes based on the target pressure and may receive feedback from one or more sensors to maintain the target pressure at the tissue interface 120.

In some embodiments, the controller 130 may have a continuous pressure mode, in which the negative-pressure source 105 is operated to provide a constant target negative pressure for the duration of treatment or until manually deactivated. Additionally or alternatively, the controller may have an intermittent pressure mode. For example, the controller 130 can operate the negative-pressure source 105 to cycle between a target pressure and atmospheric pressure. For example, the target pressure may be set at a value of −135 mmHg for a specified period of time (e.g., 5 min), followed by a specified period of time (e.g., 2 min) of deactivation. The cycle can be repeated by activating the negative-pressure source 105 which can form a square wave pattern between the target pressure and atmospheric pressure.

In some example embodiments, the increase in negative-pressure from ambient pressure to the target pressure may not be instantaneous. For example, the negative-pressure source 105 and the dressing 110 may have an initial rise time. The initial rise time may vary depending on the type of dressing and therapy equipment being used. For example, the initial rise time for one therapy system may be in a range of about 20-30 mmHg/second and in a range of about 5-10 mmHg/second for another therapy system. If the therapy system 100 is operating in an intermittent mode, the repeating rise time may be a value substantially equal to the initial rise time.

In some example dynamic pressure control modes, the target pressure can vary with time. For example, the target pressure may vary in the form of a triangular waveform, varying between a negative pressure of 50 and 135 mmHg with a rise time set at a rate of +25 mmHg/min. and a descent time set at −25 mmHg/min. In other embodiments of the therapy system 100, the triangular waveform may vary between negative pressure of 25 and 135 mmHg with a rise time set at a rate of +30 mmHg/min and a descent time set at −30 mmHg/min.

In some embodiments, the controller 130 may control or determine a variable target pressure in a dynamic pressure mode, and the variable target pressure may vary between a maximum and minimum pressure value that may be set as an input prescribed by an operator as the range of desired negative pressure. The variable target pressure may also be processed and controlled by the controller 130, which can vary the target pressure according to a predetermined waveform, such as a triangular waveform, a sine waveform, or a saw-tooth waveform. In some embodiments, the waveform may be set by an operator as the predetermined or time-varying negative pressure desired for therapy.

In some embodiments, the controller 130 may receive and process data, such as data related to instillation solution provided to the tissue interface 120. Such data may include the type of instillation solution prescribed by a clinician, the volume of fluid or solution to be instilled to a tissue site (“fill volume”), and the amount of time prescribed for leaving solution at a tissue site (“dwell time”) before applying a negative pressure to the tissue site. The fill volume may be, for example, between 10 and 500 mL, and the dwell time may be between one second to 30 minutes. The controller 130 may also control the operation of one or more components of the therapy system 100 to instill solution. For example, the controller 130 may manage fluid distributed from the solution source 145 to the tissue interface 120. In some embodiments, fluid may be instilled to a tissue site by applying a negative pressure from the negative-pressure source 105 to reduce the pressure at the tissue site, drawing solution into the tissue interface 120. In some embodiments, solution may be instilled to a tissue site by applying a positive pressure from the positive-pressure source 150 to move solution from the solution source 145 to the tissue interface 120. Additionally or alternatively, the solution source 145 may be elevated to a height sufficient to allow gravity to move solution into the tissue interface 120.

The controller 130 may also control the fluid dynamics of instillation by providing a continuous flow of solution or an intermittent flow of solution. Negative pressure may be applied to provide either continuous flow or intermittent flow of solution. The application of negative pressure may be implemented to provide a continuous pressure mode of operation to achieve a continuous flow rate of instillation solution through the tissue interface 120, or it may be implemented to provide a dynamic pressure mode of operation to vary the flow rate of instillation solution through the tissue interface 120. Alternatively, the application of negative pressure may be implemented to provide an intermittent mode of operation to allow instillation solution to dwell at the tissue interface 120. In an intermittent mode, a specific fill volume and dwell time may be provided depending, for example, on the type of tissue site being treated and the type of dressing being utilized. After or during instillation of solution, negative-pressure treatment may be applied. The controller 130 may be utilized to select a mode of operation and the duration of the negative pressure treatment before commencing another instillation cycle by instilling more solution.

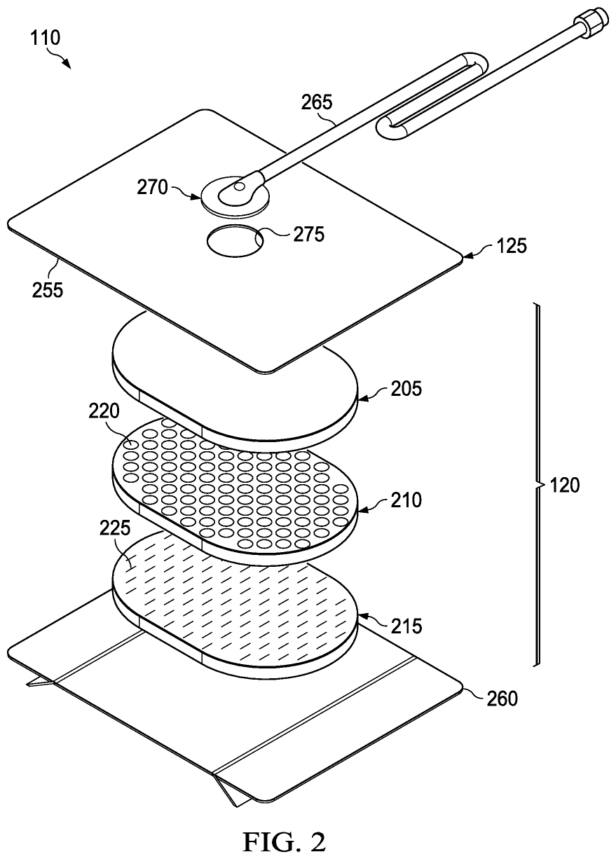

FIG. 2 is an exploded view of an example of the dressing 110 of FIG. 1, illustrating additional details that may be associated with some embodiments in which the tissue interface 120 comprises more than one layer. In the example of FIG. 2, the tissue interface 120 comprises a first layer 205, a second layer 210, and a third layer 215. In some embodiments, the first layer 205 may be disposed adjacent to the second layer 210, and the third layer 215 may also be disposed adjacent to the second layer 210 opposite the first layer 205. For example, the first layer 205, the second layer 210, and the third layer 215 may be stacked so that the first layer 205 is in contact with the second layer 210, and the second layer 210 is in contact with the first layer 205 and the third layer 215. One or more of the first layer 205, the second layer 210, and the third layer 215 may also be bonded to an adjacent layer in some embodiments.

The first layer 205 generally comprises or consists essentially of a manifold or a manifold layer, which provides a means for collecting or distributing fluid across the tissue interface 120 under pressure. For example, the first layer 205 may be adapted to receive negative pressure from a source and distribute negative pressure through multiple apertures across the tissue interface 120, which may have the effect of collecting fluid from across a tissue site and drawing the fluid toward the source. In some embodiments, the fluid path may be reversed or a secondary fluid path may be provided to facilitate delivering fluid, such as from a source of instillation solution, across the tissue interface 120.

In some illustrative embodiments, the first layer 205 may comprise a plurality of pathways, which can be interconnected to improve distribution or collection of fluids. In some illustrative embodiments, the first layer 205 may comprise or consist essentially of a porous material having interconnected fluid pathways. Examples of suitable porous material that comprise or can be adapted to form interconnected fluid pathways (e.g., channels) may include cellular foam, including open-cell foam such as reticulated foam; porous tissue collections; and other porous material such as gauze or felted mat that generally include pores, edges, and/or walls. Liquids, gels, and other foams may also include or be cured to include apertures and fluid pathways. In some embodiments, the first layer 205 may additionally or alternatively comprise projections that form interconnected fluid pathways. For example, the first layer 205 may be molded to provide surface projections that define interconnected fluid pathways. The first layer 205 may be a manifold that is substantially open to pressure and flow. In some embodiments, for example, the first layer 205 may be hydrophobic.

In some embodiments, the first layer 205 may comprise or consist essentially of a reticulated foam having pore sizes and free volume that may vary according to needs of a prescribed therapy. For example, a reticulated foam having a free volume of at least 90% may be suitable for many therapy applications, and a foam having an average pore size in a range of about 400 to about 600 microns may be particularly suitable for some types of therapy. The tensile strength of the first layer 205 may also vary according to needs of a prescribed therapy. For example, the tensile strength of a foam may be increased for instillation of topical treatment solutions. The 25% compression load deflection of the first layer 205 may be at least 0.35 pounds per square inch, and the 65% compression load deflection may be at least 0.43 pounds per square inch. In some embodiments, the tensile strength of the first layer 205 may be at least 10 pounds per square inch. The first layer 205 may have a tear strength of at least 2.5 pounds per inch. In some embodiments, the first layer 205 may be a foam comprised of polyols such as polyester or polyether, isocyanate such as toluene diisocyanate, and polymerization modifiers such as amines and tin compounds. In some embodiments, the first layer 205 may be a reticulated polyurethane foam. In some embodiments, the first layer 205 may be a reticulated polymer foam. In some examples, the first layer 205 may be a reticulated polyurethane foam such as used in GRANUFOAM™ dressing or V.A.C. VERAFLO™ dressing, both available from KCI of San Antonio, Tex.

Other suitable materials for the first layer 205 may include non-woven fabrics (Libeltex, Freudenberg), three-dimensional (3D) polymeric structures (molded polymers, embossed and formed films, and fusion bonded films [Supracore]), and mesh, for example.

In some examples, the first layer 205 may include a 3D textile, such as various textiles commercially available from Baltex, Muller, and Heathcoates. A 3D textile of polyester fibers may be particularly advantageous for some embodiments. For example, the first layer 205 may comprise or consist essentially of a three-dimensional weave of polyester fibers. In some embodiments, the fibers may be elastic in at least two dimensions. A puncture-resistant fabric of polyester and cotton fibers having a weight of about 650 grams per square meter and a thickness of about 1-2 millimeters may be particularly advantageous for some embodiments. Such a puncture-resistant fabric may have a warp tensile strength in a range of about 330 to about 340 kilograms and a weft tensile strength in a range of about 270 to about 280 kilograms in some embodiments. Another particularly suitable material may be a polyester spacer fabric having a weight of about 470 grams per square meter, which may have a thickness in a range of about 4 to about 5 millimeters in some embodiments. Such a spacer fabric may have a compression strength in a range of about 20 to about 25 kilopascals (at 40% compression). Additionally or alternatively, the first layer 205 may comprise or consist of a material having substantial linear stretch properties, such as a polyester spacer fabric having 2-way stretch and a weight of about 380 grams per square meter. A suitable spacer fabric may have a thickness in a range of about 3 to about 4 millimeters, and may have a warp and weft tensile strength in a range of about 30 to about 40 kilograms in some embodiments. The fabric may have a close-woven layer of polyester on one or more opposing faces in some examples. In some embodiments, a woven layer may be advantageously disposed on a first layer 205 to face a tissue site.

The first layer 205 generally has a first planar surface and a second planar surface opposite the first planar surface. The thickness of the first layer 205 between the first planar surface and the second planar surface may also vary according to needs of a prescribed therapy. For example, the thickness of the first layer 205 may be decreased to relieve stress on other layers and to reduce tension on peripheral tissue. The thickness of the first layer 205 can also affect the conformability of the first layer 205. In some embodiments, the first layer 205 may have a thickness in a range of about 2 millimeters to about 10 millimeters. In some embodiments, for example, the first layer 205 may have a thickness less than 12 millimeters. In some embodiments, for example, the first layer 205 may have a thickness less than 10 millimeters. In some embodiments, for example, the first layer 205 may have a thickness less than 5 millimeters. For example only and without limitation, in some embodiments, the first layer 205 may be unfelted GRANUFOAM™ having a thickness in a range of about 6 millimeters to about 10 millimeters. In other embodiments, the first layer 205 may have a thickness in a range of about 2 millimeters to about 5 millimeters. For example only and without limitation, in some embodiments, the first layer 205 may be felted GRANUFOAM™ having a thickness in a range of about 2 millimeters to about 5 millimeters.

The second layer 210 may comprise or consist essentially of a support layer for third layer 215. In some embodiments, the second layer 210 may comprise or consist essentially of a closed-cell foam. For example, the second layer 210 may comprise or consist essentially of silicone, polyurethane (PU), or ethylene vinyl acetate (EVA). For example, the second layer 210 may be a closed-cell foam having an average pore size in a range of about 0.2 millimeters (200 microns) to about 1 millimeter (1000 microns). In some embodiments, the second layer 210 may be a closed-cell foam having a porosity in a range of about 200 ppi to about 30 ppi.

The second layer 210 generally has a first planar surface and a second planar surface opposite the first planar surface. The thickness of the second layer 210 between the first planar surface and the second planar surface may also vary according to needs of a prescribed therapy. In some embodiments, the second layer 210 may have a thickness in a range of about 0.5 millimeters to about 10 millimeters. In some embodiments, the second layer 210 may have a thickness in a range of about 1 millimeter to about 3 millimeters. In some embodiments, the second layer 210 may have a hardness or durometer in a range of about 10 Shore A to about 50 Shore A. The face of the second layer 210 may have a smooth surface finish either due to the original manufacturing process or due to a post-formation process.

In some embodiments, the second layer 210 may be hydrophobic, comprised of hydrophobic materials, and/or treated to be hydrophobic. The hydrophobicity of the second layer 210 may vary, but may have a contact angle with water of at least ninety degrees in some embodiments. In some embodiments the second layer 210 may have a contact angle with water of no more than 150 degrees. For example, in some embodiments, the contact angle of the second layer 210 may be in a range of at least 90 degrees to about 120 degrees, or in a range of at least 120 degrees to about 150 degrees. Water contact angles can be measured using any standard apparatus. Although manual goniometers can be used to visually approximate contact angles, contact angle measuring instruments can often include an integrated system involving a level stage, liquid dropper such as a syringe, camera, and software designed to calculate contact angles more accurately and precisely, among other things. Non-limiting examples of such integrated systems may include the FTÅ125, FTÅ200, FTÅ2000, and FTÅ4000 systems, all commercially available from First Ten Angstroms, Inc., of Portsmouth, Va., and the DTA25, DTA30, and DTA100 systems, all commercially available from Kruss GmbH of Hamburg, Germany. Unless otherwise specified, water contact angles herein are measured using deionized and distilled water on a level sample surface for a sessile drop added from a height of no more than 5 cm in air at 20-25° C. and 20-50% relative humidity. Contact angles reported herein represent averages of 5-9 measured values, discarding both the highest and lowest measured values. The hydrophobicity of the second layer 210 may be further enhanced with a hydrophobic coating of other materials, such as silicones and fluorocarbons, either as coated from a liquid, or plasma coated.

The second layer 210 may also be suitable for coupling to other layers, including the first layer 205 and/or the third layer 215. In some embodiments, for example, the second layer 210 may be coupled to other layers by welding, bonding, adhering, or laminating. For example, the second layer 210 may be adapted for welding to polyurethane foams using heat, radio frequency (RF) welding, or other methods to generate heat such as ultrasonic welding. RF welding may be particularly suitable for more polar materials, such as polyurethane, polyamides, polyesters and acrylates. Sacrificial polar interfaces may be used to facilitate RF welding of less polar film materials, such as polyethylene. In some embodiments, the second layer may be bonded to other layers, including the first layer 205 and/or the third layer 205, using adhesives.

The area density of the second layer 210 may vary according to a prescribed therapy or application. In some embodiments, an area density of less than 40 grams per square meter may be suitable, and an area density of about 20-30 grams per square meter may be particularly advantageous for some applications.

As illustrated in the example of FIG. 2, the second layer 210 may have one or more apertures 220. The apertures 220 may be formed by cutting, perforating, punching, or by other suitable techniques for forming an aperture, opening, perforation, or hole in the second layer 210, including but not limited to using a single- or multiple-blade cutter, a laser, a water jet, a hot knife, a computer numeric control (CNC) cutter, a hot wire, local RF or ultrasonic energy, and/or a single- or multiple-punch tool. The apertures 220 extend from the first planar surface to the second planar surface of the second layer 210, creating a through hole or passage in the second layer 210. The apertures 220 in the second layer 210 may have many shapes, for example, including but not limited to circles, squares, stars, ovals, polygons, slits, complex curves, rectilinear shapes, triangles or may have some combination of such shapes.

Each of the apertures 220 may have uniform or similar geometric properties. For example, in some embodiments, each of the apertures 220 may be circular apertures, having substantially the same diameter. In some embodiments, each of the apertures 220 may have a diameter in a range of about 1 millimeter to about 50 millimeters. In other embodiments, each of the apertures 220 may have a diameter in a range of about 1 millimeter to about 20 millimeters. In other embodiments, each of the apertures 220 may have a diameter in a range of about 1 millimeter to about 5 millimeters. In yet other embodiments, each of the apertures 220 may have a diameter in a range of about 2 millimeters to about 3 millimeters.

The third layer 215 may comprise or consist essentially of a means for controlling or managing fluid flow. The third layer 215 may be considered a fluid control layer. In some embodiments, the third layer 215 may comprise or consist essentially of a closed cell foam. For example, the third layer 215 may comprise or consist essentially of silicone, polyurethane (PU), or ethylene vinyl acetate (EVA). The structure of these closed-cell foams can provide a surface that interacts little, if any, with biological tissues and fluids, providing a surface that may encourage the free flow of liquids and low adherence, which can be particularly advantageous for many applications. For example, the third layer 215 may be a closed cell foam having an average pore size in a range of about 0.2 millimeters (200 microns) to about 1 millimeter (1000 microns). In some embodiments, the third layer 215 may be a closed-cell foam having a porosity in a range of about 200 ppi to about 30 ppi.

The third layer 215 generally has a first planar surface and a second planar surface opposite the first planar surface. The thickness of the third layer 215 between the first planar surface and the second planar surface may also vary according to needs of a prescribed therapy. In some embodiments, the third layer 215 may have a thickness in a range of about 0.5 millimeters to about 10 millimeters. In some embodiments, the third layer 215 may have a thickness in a range of about 1 millimeter to about 3 millimeters. In some embodiments, the third layer 215 may have a hardness or durometer in a range of about 10 Shore A to about 50 Shore A.

Further, the third layer 215 may be hydrophobic, comprised of hydrophobic materials, and/or treated to be hydrophobic. In some embodiments, the third layer 215 may be highly hydrophobic. In some embodiments, the third layer 215 may be more hydrophobic than the second layer 210. The hydrophobicity of the third layer 215 may vary, but may have a contact angle with water of at least ninety degrees in some embodiments. In some embodiments the third layer 215 may have a contact angle with water of no more than 150 degrees. For example, in some embodiments, the contact angle of the third layer 215 may be in a range of at least 90 degrees to about 120 degrees, or in a range of at least 120 degrees to about 150 degrees. Water contact angles can be measured using any standard apparatus. Although manual goniometers can be used to visually approximate contact angles, contact angle measuring instruments can often include an integrated system involving a level stage, liquid dropper such as a syringe, camera, and software designed to calculate contact angles more accurately and precisely, among other things. Non-limiting examples of such integrated systems may include the FTÅ125, FTÅ200, FTÅ2000, and FTÅ4000 systems, all commercially available from First Ten Angstroms, Inc., of Portsmouth, Va., and the DTA25, DTA30, and DTA100 systems, all commercially available from Kruss GmbH of Hamburg, Germany. Unless otherwise specified, water contact angles herein are measured using deionized and distilled water on a level sample surface for a sessile drop added from a height of no more than 5 cm in air at 20-25° C. and 20-50% relative humidity. Contact angles reported herein represent averages of 5-9 measured values, discarding both the highest and lowest measured values. The hydrophobicity of the third layer 215 may be further enhanced with a hydrophobic coating of other materials, such as silicones and fluorocarbons, either as coated from a liquid, or plasma coated.

As illustrated in the example of FIG. 2, the third layer 215 may have one or more fluid restrictions 225, which can be distributed across the third layer 215 such that they are aligned or registered with the one or more apertures 220 in the second layer 210. The fluid restrictions 225 may be bi-directional and pressure-responsive. For example, the fluid restrictions 225 generally may comprise or consist essentially of an elastic passage that is normally unstrained to substantially reduce liquid flow, and can expand in response to a pressure gradient. In some embodiments, the fluid restrictions 225 may comprise or consist essentially of perforations in the third layer 215. Perforations may be formed by removing material from the third layer 215. For example, perforations may be formed by cutting through the third layer 215. In the absence of a pressure gradient across the perforations, the passages may be sufficiently small to form a seal or flow restriction, which can substantially reduce or prevent liquid flow. Additionally or alternatively, one or more of the fluid restrictions 225 may be an elastomeric valve that is normally closed when unstrained to substantially prevent liquid flow, and can open in response to a pressure gradient. A fenestration in the third layer 215 may be a suitable valve for some applications. Fenestrations may also be formed by removing material from the third layer 215, but the amount of material removed and the resulting dimensions of the fenestrations may be up to an order of magnitude less than perforations. A slit in the third layer 215 may be a suitable valve for some applications.

For example, some embodiments of the fluid restrictions 225 may comprise or consist essentially of one or more slits, slots or combinations of slits and slots in the third layer 215. In some embodiments, the fluid restrictions 225 may comprise or consist of linear slots having a length less than about 5 millimeters and a width less than about 2 millimeters. The length may be at least about 2 millimeters, and the width may be at least about 0.3 millimeters in some embodiments. In some embodiments, the fluid restrictions 225 may have a width in a range of about 0.3 millimeters to about 0.7 millimeters and may have a length in a range of about 2 millimeters to about 3 millimeters. In other embodiments, the fluid restrictions 225 may have a width in a range of about 0.5 millimeters to about 1 millimeter and may have a length in a range of about 2 millimeters to about 10 millimeters. For example, a length of about 3 millimeters and a width of about 0.8 millimeters may be particularly suitable for many applications, and a tolerance of about 0.1 millimeter may also be acceptable. Such dimensions and tolerances may be achieved with a laser cutter, for example. Slots of such configurations may function as imperfect valves that substantially reduce liquid flow in a normally closed or resting state. For example, such slots may form a flow restriction without being completely closed or sealed. The slots can expand or open wider in response to a pressure gradient to allow increased liquid flow. The slots may be configured to permit fluid flow and inhibit exposure of the first layer to the tissue site.

In some embodiments, for example only and without limitation, the dressing 110 may have a thickness greater than 1 millimeter. In some embodiments, for example, the dressing 110 may have a thickness greater than 2 millimeters. In some embodiments, for example, the dressing 110 may have a thickness greater than 5 millimeters. In some embodiments, for example, the dressing 110 may have a thickness greater than 6 millimeters. In some embodiments, for example, the dressing 110 may have a thickness greater than 5 millimeters. In some embodiments, for example, the dressing 110 may have a thickness greater than 10 millimeters.

In the example of FIG. 2, the dressing 110 may further include an attachment device, such as an adhesive 255. The adhesive 255 may be, for example, a medically-acceptable, pressure-sensitive adhesive that extends about a periphery, a portion, or the entire cover 125. In some embodiments, the adhesive 255 may be disposed in a margin of the cover 125 that extends beyond the first layer 205, the second layer 210, and the third layer 215. In some embodiments, for example, the adhesive 255 may be an acrylic adhesive having a coating weight in a range of about 25 to about 65 grams per square meter (g.s.m.). Thicker adhesives, or combinations of adhesives, may be applied in some embodiments to improve the seal and reduce leaks. In some embodiments, such a layer of the adhesive 255 may be continuous or discontinuous. Discontinuities in the adhesive 255 may be provided by apertures or holes (not shown) in the adhesive 255. The apertures or holes in the adhesive 255 may be formed after application of the adhesive 255 or by coating the adhesive 255 in patterns on a carrier layer, such as, for example, a side of the cover 125. Apertures or holes in the adhesive 255 may also be sized to enhance the MVTR of the dressing 110 in some example embodiments.

As illustrated in the example of FIG. 2, in some embodiments, the dressing 110 may include a release liner 260 attached to or positioned adjacent to the third layer 215 to protect the adhesive 255 prior to use. The release liner 260 may also provide stiffness to assist with, for example, deployment of the dressing 110. The release liner 260 may be, for example, a casting paper, a film, or polyethylene. Further, in some embodiments, the release liner 260 may be a polyester material such as polyethylene terephthalate (PET), or similar polar semi-crystalline polymer. The use of a polar semi-crystalline polymer for the release liner 260 may substantially preclude wrinkling or other deformation of the dressing 110. For example, the polar semi-crystalline polymer may be highly orientated and resistant to softening, swelling, or other deformation that may occur when brought into contact with components of the dressing 110, or when subjected to temperature or environmental variations, or sterilization. In some embodiments, the release liner 260 may have a surface texture that may be imprinted on an adjacent layer, such as the third layer 215. Further, a release agent may be disposed on a side of the release liner 260 that is configured to contact the third layer 215. For example, the release agent may be a silicone coating and may have a release factor suitable to facilitate removal of the release liner 260 by hand and without damaging or deforming the dressing 104. In some embodiments, the release agent may be a fluorocarbon or a fluorosilicone, for example. In other embodiments, the release liner 260 may be uncoated or otherwise used without a release agent.

FIG. 2 also illustrates one example of a fluid conductor 265 and a dressing interface 270. As shown in the example of FIG. 2, the fluid conductor 265 may be a flexible tube, which can be fluidly coupled on one end to the dressing interface 270. The dressing interface 270 may be an elbow connector, as shown in the example of FIG. 2, which can be placed over an aperture 275 in the cover 125 to provide a fluid path between the fluid conductor 265 and the tissue interface 120.

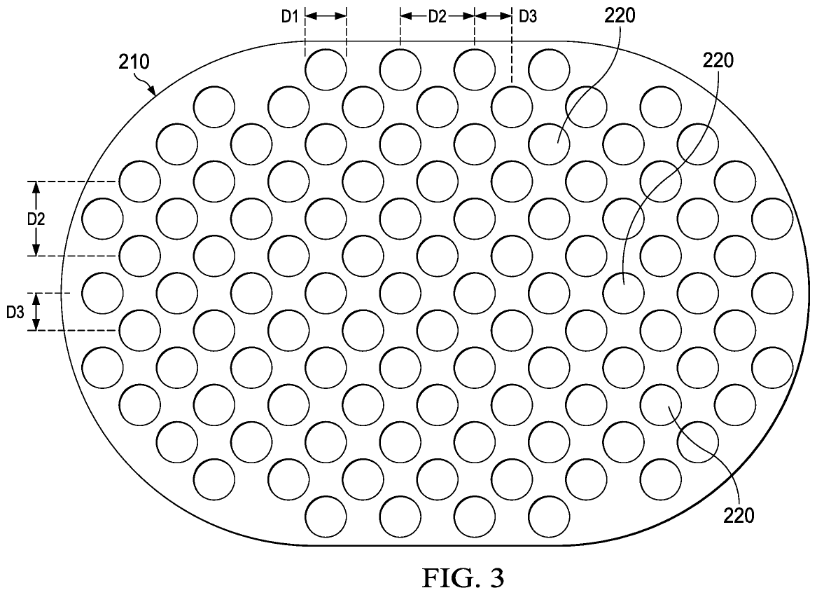

FIG. 3 is a schematic view of an example configuration of the second layer 210, illustrating additional details that may be associated with some embodiments. The second layer 210 is shown as having a stadium shape; however, in other embodiments the second layer 210 may have other shapes including but not limited to, triangular, rectangular, rectilinear, square, pentagonal, hexagonal, octagonal, circular, ovular, and elliptical. In the example of FIG. 3, the apertures 220 are generally circular and have a diameter D1, which may range from about 2 millimeters to about 10 millimeters in some embodiments. In some embodiments, for example, the diameter D1 of the apertures 220 may range from about 2 millimeters to about 3 millimeters. A diameter D1 of about 3 millimeters may be particularly suitable for some embodiments. In some embodiments, for example, the diameter D1 of the apertures 220 may be less than 5 millimeters. In some embodiments, for example, the diameter D1 of the apertures 220 may be less than 4 millimeters. In some embodiments, for example, the diameter D1 of the apertures 220 may be less than 3 millimeters. In some embodiments, for example, the diameter D1 of the apertures 220 may be less than 2 millimeters. In some embodiments, for example, the diameter D1 of the apertures 220 may be greater than 3 millimeters. In some embodiments, for example, the diameter D1 of the apertures 220 may be greater than 10 millimeters. The apertures 220 are shown as having a circular shape; however, in other embodiments the apertures 220 may have other shapes including but not limited to, triangular, rectangular, rectilinear, square, pentagonal, hexagonal, octagonal, ovular, and elliptical.

FIG. 3 also illustrates an example of a uniform distribution pattern of the apertures 220. In FIG. 3, the apertures 220 are substantially coextensive with the second layer 210, and are distributed across the second layer 210 in a grid of parallel rows and columns. Within each row and column, the apertures 220 may be equidistant from each other, as illustrated in the example of FIG. 3. FIG. 3 illustrates one example configuration that may be particularly suitable for many applications, in which the centers of apertures 220 are (i) spaced a distance D2 apart along alternating rows and alternating columns and (ii) are offset D3 between each row and each column. That is, the rows and columns of apertures 220 are staggered or offset. However, it will be understood that in other embodiments, for example, the rows and columns of apertures 220 are not staggered or offset, but are aligned. In some examples, the distance D2 may range from about 3 millimeters to about 20 millimeters, and the offset D3 may range from about 1 millimeter to about 10 millimeters. In some embodiments, for example, the distance D2 may be less than 3 millimeters. In some embodiments, for example, the distance D2 may be greater than 20 millimeters. In some embodiments, for example, the offset D3 may be less than 1 millimeter. In some embodiments, for example, the offset D3 may be greater than 10 millimeters. In some embodiments, the distance D2 is twice the offset D3. The dimension and/or spacing of the apertures 220 may vary in some embodiments to increase the density of the apertures 220 according to therapeutic requirements.

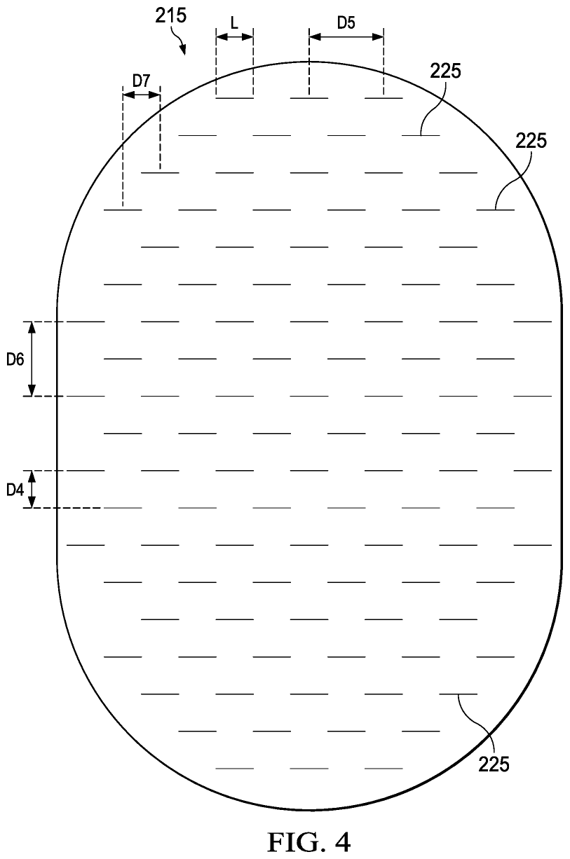

FIG. 4 is a schematic view of an example of the third layer 215, illustrating additional details that may be associated with some embodiments. The third layer 215 is shown as having a stadium shape; however, in other embodiments the third layer 215 may have other shapes including but not limited to, triangular, rectangular, rectilinear, square, pentagonal, hexagonal, octagonal, circular, ovular, and elliptical. As illustrated in the example of FIG. 4, the fluid restrictions 225 may each consist essentially of one or more slits having a length L, which may range from about 2 millimeters to about 10 millimeters in some embodiments. In some embodiments, for example, the length L of the fluid restrictions 225 may be in a range of about 2 millimeters to about 3 millimeters. A length L of about 3 millimeters may be particularly suitable for some embodiments. In some embodiments, for example, the length L may be less than 2 millimeters. In some embodiments, for example, the length L may be greater than 10 millimeters. In some embodiments, for example, there is a relationship between the thickness of the third layer 215 and the length L of the fluid restrictions 225. For example, where the third layer 215 is about 2 millimeters thick, the length L of the fluid restrictions 225 may be about 3 millimeters. If however the third layer 215 is about 3 millimeters thick, the length L of the fluid restrictions 225 may be increased by about 0.5 millimeters to about 3.5 millimeters. Thus, in some embodiments, for example, the length L of the fluid restrictions 225 may increase by 0.5 millimeters for every 1 millimeter increase in the thickness of the third layer 215 above 3 millimeters.

FIG. 4 additionally illustrates an example of a uniform distribution pattern of the fluid restrictions 225. In FIG. 4, the fluid restrictions 225 are substantially coextensive with the third layer 215, and are distributed across the third layer 215 in a grid of parallel rows and columns, in which the fluid restrictions 225 are also mutually parallel to each other. In some embodiments, the rows may be spaced a distance D4, which may range from about 2 millimeters to about 10 millimeters in some embodiments. A distance D4 of about 3 millimeters on center may be suitable for some embodiments. In some embodiments, for example, the distance D4 may be less than 2 millimeters. In some embodiments, for example, the distance D4 may be greater than 10 millimeters. The fluid restrictions 225 within each of the rows may be spaced a distance D5, which may range from about 2 millimeters to about 10 millimeters on center in some embodiments. A distance D5 of about 6 millimeters on center may be suitable for some embodiments. In some embodiments, for example, the distance D5 may be less than 2 millimeters. In some embodiments, for example, the distance D5 may be greater than 10 millimeters. The fluid restrictions 225 are aligned in alternating rows and separated by a distance D6, which may range from about 2 millimeters to about 10 millimeters in some embodiments. A distance D6 of about 6 millimeters may be suitable for some embodiments. In some embodiments, for example, the distance D6 may be less than 2 millimeters. In some embodiments, for example, the distance D6 may be greater than 10 millimeters. In some embodiments, the distance D6 is twice the distance D4. The fluid restrictions 225 in adjacent rows may be aligned or offset in some embodiments. For example, the centers of the fluid restrictions 225 in adjacent rows may be offset D7 between each row and column, as illustrated in FIG. 4. In some embodiments, the offset D7 may range from about 1 millimeter to about 5 millimeters. An offset D7 of about 2 millimeters may be suitable for some embodiments. In some embodiments, for example, the offset D7 may be less than 1 millimeter. In some embodiments, for example, the offset D7 may be greater than 5 millimeters. While the rows of fluid restrictions 225 may be staggered or offset in some embodiments, it will be understood that in other embodiments, for example, the rows of fluid restrictions 225 are not staggered or offset, but are aligned. The dimensions and/or spacing of the fluid restrictions 225 may vary in some embodiments to increase the density of the fluid restrictions 225 according to therapeutic requirements. In some embodiments, the fluid restrictions 225 may be sized and/or spaced (e.g., dimensions L, D4, D5, D6, and/or D7 may be selected) to achieve an open area in a range of about 8% to about 10% when subjected to negative pressure.

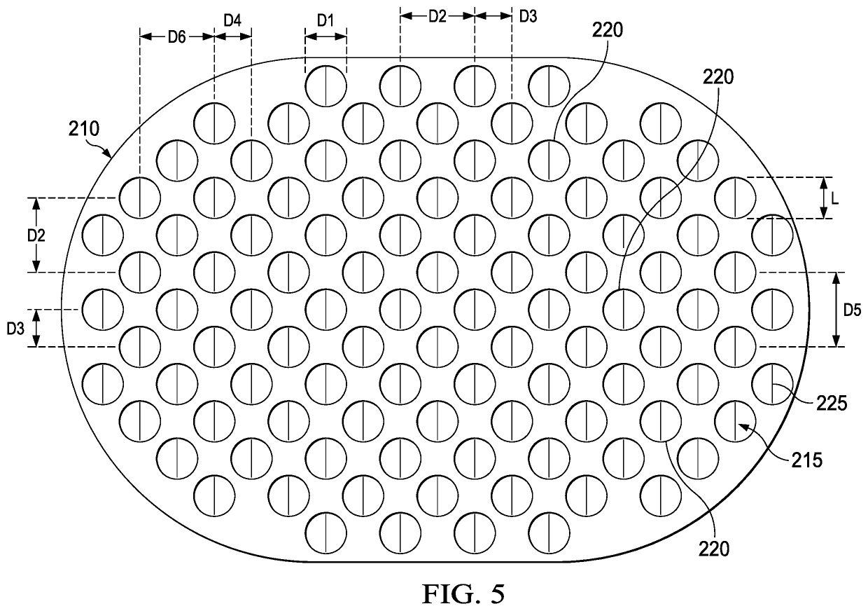

FIG. 5 is a schematic view of the apertures 220 in the second layer 210 of FIG. 3 overlaid and registered with the fluid restrictions 225 in the third layer 215 of FIG. 4, illustrating additional details that may be associated with some example embodiments of the tissue interface 120. For example, as illustrated in FIG. 5, the fluid restrictions 225 may be aligned, overlapping, in registration with, or otherwise fluidly coupled to the apertures 220 in some embodiments. Thus, at least some of the plurality of fluid restrictions 225 have a corresponding aperture 220, wherein the corresponding fluid restrictions 225 and apertures 220 are in fluid communication. The apertures 220 in the example of FIG. 5 are generally sized and configured so that one fluid restriction 225 is registered with each one of the apertures 220. For example, in some embodiments, the length L of each of the fluid restrictions 225 may be equal to the diameter D1 of each of the apertures 220. In some embodiments, the fluid restrictions 225 have an average length that does not substantially exceed an average dimension of the apertures 220. In other embodiments, the length L of each of the fluid restrictions 225 may be smaller than the diameter D1 of each of the apertures 220. In yet other embodiments, the length L of each of the fluid restrictions 225 may be greater than the diameter D1 of each of the apertures 220. Accordingly, in embodiments where the dimensions of the fluid restrictions 225 exceed the dimensions of the apertures 220, the size of the apertures 220 may limit the deflection of the third layer 215 adjacent the fluid restrictions 225 into the apertures 220 in the second layer 210. Additionally, the size of the apertures 220 may define, control, or limit the dimension of the deflected fluid restrictions 225 when a negative pressure gradient is applied across the fluid restrictions 225. Thus, the apertures 220 may limit the effective size of the fluid restrictions 225. For example, the diameter D1 of the apertures 220 may be sized such that there is deflection of about 1 millimeter of the third layer 215 proximate the fluid restrictions 225 into and away from the apertures 220 in the second layer 210. In some embodiments, each of the apertures 220 are sized to expose no more than two of the fluid restrictions 225. Additionally, as shown in the example of FIG. 5, distance D2 may be equal to distance D6. In some embodiments, more than one fluid restriction 225 may be registered to each aperture 220. That is, for example, multiple fluid restrictions 225 may be aligned with one of the apertures 220. In some embodiments, one or more of the fluid restrictions 225 may be only partially registered with the apertures 220. In other examples, one or more of the fluid restrictions 225 may be registered with more than one of the apertures 220. For example, any one or more of the fluid restrictions 225 may be a perforation or a fenestration that extends across two or more of the apertures 220. Additionally or alternatively, one or more of the fluid restrictions 225 may not be registered with any of the apertures 220.

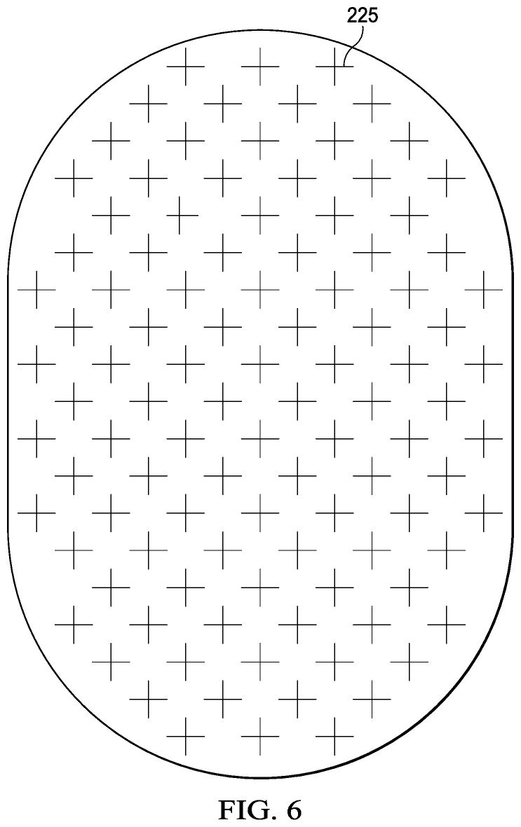

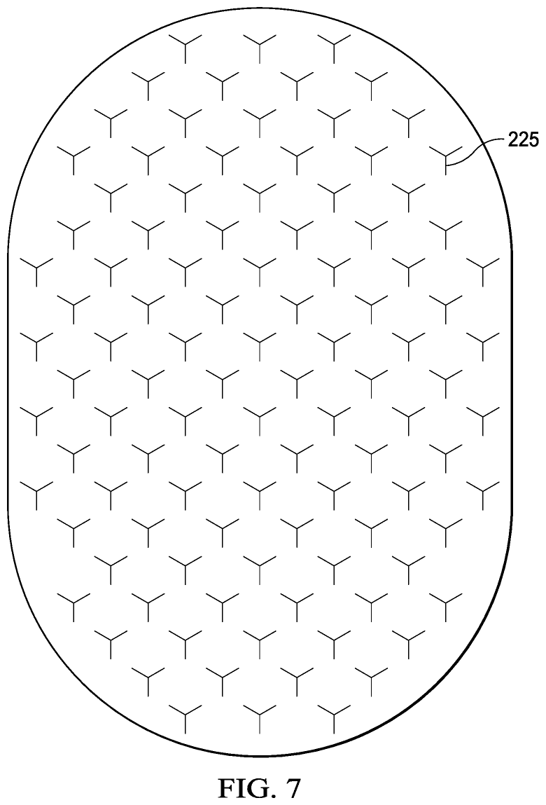

FIG. 6 and FIG. 7 illustrate other example configurations of the fluid restrictions 225, in which the fluid restrictions 225 each generally comprise a combination of intersecting slits or cross-slits.

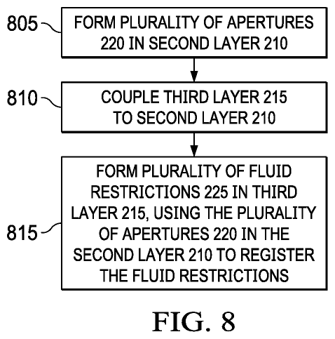

FIG. 8 is a flow diagram, illustrating an example method of manufacturing the second layer 210 and the third layer 215. At step 805, apertures 220 are formed in the second layer 210. For example, the apertures 220 may be formed by cutting, perforating, punching, or by other suitable techniques for forming an aperture, opening, perforation, or hole in the second layer 210, including but not limited to using a single- or multiple-blade cutter, a laser, a water jet, a hot knife, a computer numeric control (CNC) cutter, a hot wire, local RF or ultrasonic energy, and/or a single- or multiple-punch tool. In some embodiments, the apertures 220 may be formed one aperture 220 at a time. In other embodiments, multiple apertures 220 may be formed at a time. In yet other embodiments, all apertures 220 may be formed at the same time. At step 810, third layer 215 is coupled to second layer 210. For example only and without limitation, the third layer 215 may be coupled to the second layer 210 using heat, radio frequency (RF) welding, ultrasonic welding, adhesives, and/or mechanical fasteners. At step 815, fluid restrictions 225 are formed in third layer 215, using the apertures 220 in the second layer 210 to register the fluid restrictions 225. For example, the fluid restrictions 225 may be formed by cutting, perforating, punching, or by other suitable techniques for forming an opening, perforation, fenestration, or slit in the third layer 215, including but not limited to using a single- or multiple-blade cutter, a laser, a water jet, a hot knife, a computer numeric control (CNC) cutter, a hot wire, local RF or ultrasonic energy, and/or a single- or multiple-punch tool. In some embodiments, the fluid restrictions 225 may be formed one fluid restriction 225 at a time. In other embodiments, multiple fluid restrictions 225 may be formed at a time. In yet other embodiments, all fluid restrictions 225 may be formed at the same time. By forming the fluid restrictions 225 after the third layer 215 is coupled to the second layer 210, the apertures 220 in the second layer 210 can be used to align and locate the fluid restrictions 225 with respect to the apertures 220. That is, the apertures 220 may be used as a jig or template for forming the fluid restrictions 225. For example, in some embodiments, a cutting knife may be extended into and through the apertures 220 in the second layer 210 to cut the fluid restrictions 225 in the third layer 215, wherein the apertures 220 are used to locate the cutting knife during the fluid restriction 225 forming operation.

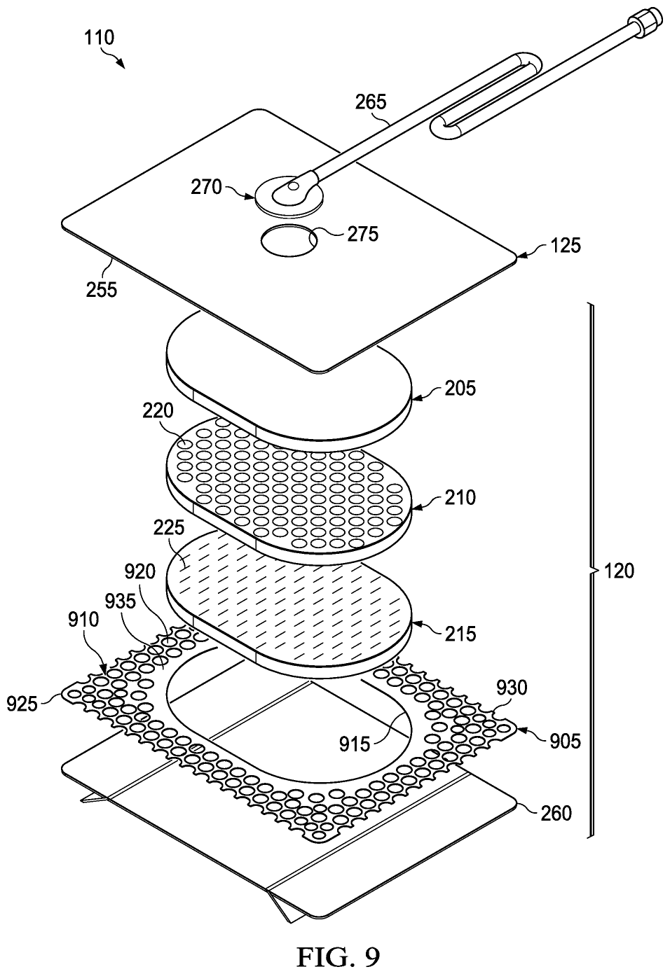

FIG. 9 illustrates an additional embodiment of dressing 110 which further includes a fourth layer 905. The fourth layer 905 may comprise or consist essentially of a sealing layer formed from a soft, pliable material suitable for providing a fluid seal with a tissue site, such as a suitable gel material, and may have a substantially flat surface. For example, the fourth layer 905 may comprise, without limitation, a silicone gel, a soft silicone, hydrocolloid, hydrogel, polyurethane gel, polyolefin gel, hydrogenated styrenic copolymer gel, a foamed gel, a soft closed cell foam such as polyurethanes and polyolefins coated with an adhesive, polyurethane, polyolefin, or hydrogenated styrenic copolymers. In some embodiments, the fourth layer 905 may have a thickness in a range of about 200 microns to about 1000 microns. In some embodiments, the fourth layer 905 may have a hardness between about 5 Shore OO and about 80 Shore OO. Further, the fourth layer 905 may be comprised of hydrophobic or hydrophilic materials.

In some embodiments, the fourth layer 905 may be a hydrophobic-coated material. For example, the fourth layer 905 may be formed by coating a spaced material, such as, for example, woven, nonwoven, molded, or extruded mesh with a hydrophobic material. The hydrophobic material for the coating may be a soft silicone, for example.

The fourth layer 905 may have a periphery 910 surrounding or around a treatment aperture 915, and apertures 920 in the periphery 910 disposed around the treatment aperture 915. The treatment aperture 915 may be complementary or correspond to a surface area of the third layer 215 in some examples. For example, the treatment aperture 915 may form a frame, window, or other opening around a surface of the third layer 215. The fourth layer 905 may also have corners 925 and edges 930. The corners 925 and the edges 930 may be part of the periphery 910. The fourth layer 905 may have an interior border 935 around the treatment aperture 915, which may be substantially free of the apertures 920, as illustrated in the example of FIG. 9. In some examples, as illustrated in FIG. 9, the treatment aperture 915 may be symmetrical and centrally disposed in the fourth layer 905, forming an open central window.

For example, the apertures 920 may be formed by cutting, perforating, punching, or by other suitable techniques for forming an aperture, opening, perforation, or hole in the fourth layer 905, including but not limited to using a single- or multiple-blade cutter, a laser, a water jet, a hot knife, a computer numeric control (CNC) cutter, a hot wire, local RF or ultrasonic energy, and/or a single- or multiple-punch tool. The apertures 920 may have a uniform distribution pattern, or may be randomly distributed on the fourth layer 905. The apertures 920 in the fourth layer 905 may have many shapes, including circles, squares, stars, ovals, polygons, slits, complex curves, rectilinear shapes, triangles, for example, or may have some combination of such shapes.

Each of the apertures 920 may have uniform or similar geometric properties. For example, in some embodiments, each of the apertures 920 may be circular apertures, having substantially the same diameter. In some embodiments, each of the apertures 920 may have a diameter in a range of about 1 millimeter to about 50 millimeters. In other embodiments, the diameter of each of the apertures 920 may be in a range of about 1 millimeter to about 20 millimeters. In some embodiments, for example, the diameter may be less than 1 millimeter. In some embodiments, for example, the diameter may be greater than 50 millimeters.

In other embodiments, geometric properties of the apertures 920 may vary. For example, the diameter of the apertures 920 may vary depending on the position of the apertures 920 in the fourth layer 905. For example, in some embodiments, the apertures 920 disposed in the periphery 910 may have a diameter in a range of about 5 millimeters to about 10 millimeters. A range of about 7 millimeters to about 9 millimeters may be suitable for some examples. In some embodiments, the apertures 920 disposed in the corners 925 may have a diameter in a range of about 7 millimeters to about 8 millimeters. In some embodiments, for example, the diameter may be less than 5 millimeter. In some embodiments, for example, the diameter may be greater than 10 millimeters.

At least one of the apertures 920 in the periphery 910 of the fourth layer 905 may be positioned at the edges 930 of the periphery 910, and may have an interior cut open or exposed at the edges 930 that is in fluid communication in a lateral direction with the edges 930. The lateral direction may refer to a direction toward the edges 930 and in the same plane as the fourth layer 905. As shown in the example of FIG. 9, the apertures 920 in the periphery 910 may be positioned proximate to or at the edges 930 and in fluid communication in a lateral direction with the edges 930. The apertures 920 positioned proximate to or at the edges 930 may be spaced substantially equidistant around the periphery 910 as shown in the example of FIG. 9. Alternatively, the spacing of the apertures 920 proximate to or at the edges 930 may be irregular.

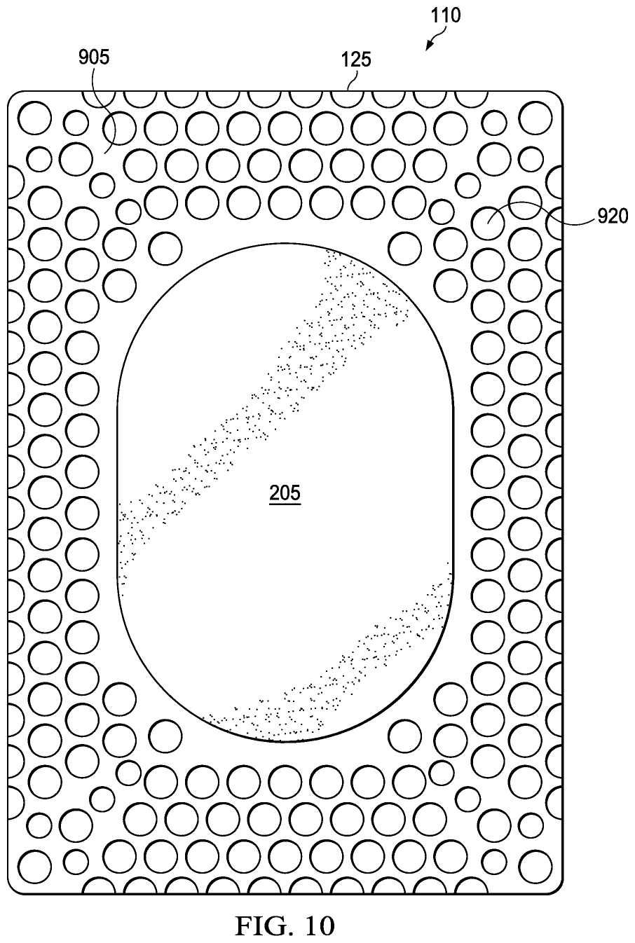

FIG. 10 is a top view of the dressing 110 in the example of FIG. 9, as assembled, illustrating additional details that may be associated with some embodiments. As illustrated in the example of FIG. 9, the cover 125 and the fourth layer 905 may have substantially the same perimeter shape and dimensions, so that the cover 125 and the fourth layer 905 are coextensive in some examples. The cover 125 may be substantially transparent, allowing visibility of the apertures 920 in some embodiments. The third layer 215 may be centrally disposed over the fourth layer 905, such as over the treatment aperture 915 (not visible in FIG. 10). The second layer 210 may be disposed over and coupled to the third layer 210. The first layer 205 may be disposed over and coupled to the second layer 210. The cover 125 may be disposed over the first layer 205 and coupled to the fourth layer 905 around the first layer 205 so that at least some of the adhesive 255 can be disposed adjacent to the apertures 920.

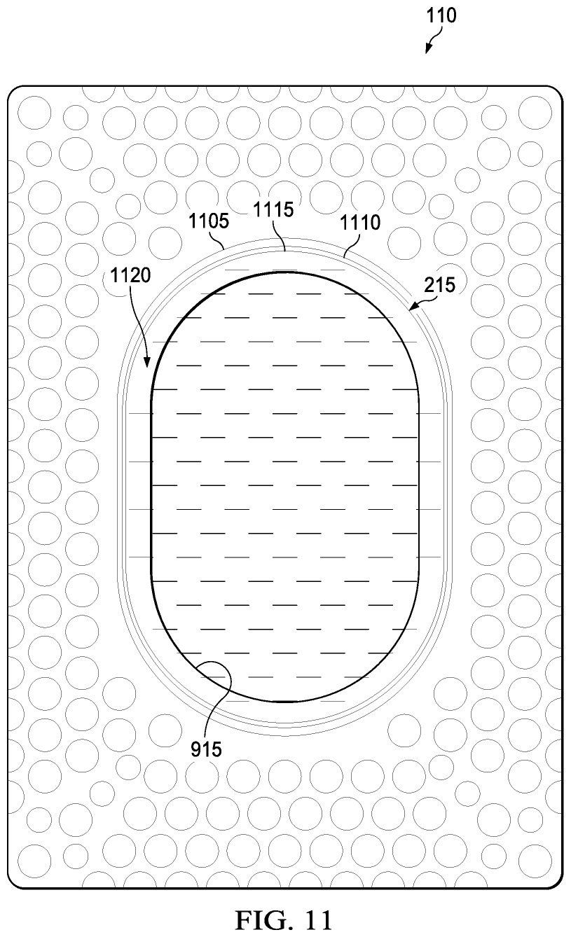

FIG. 11 is a bottom view of the dressing 110 in the example of FIG. 9, as assembled, illustrating additional details that may be associated with some embodiments. As illustrated in the example of FIG. 11, a substantial number of the fluid restrictions 225 may be aligned or otherwise exposed through the treatment aperture 915. In some embodiments, the first layer 205, the second layer 210, and the third layer 215 may be substantially aligned with the treatment aperture 915, or may extend across the treatment aperture 915.

Additionally, the first layer 205 may have a first edge 1105, the second layer 210 may have a second edge 1110, and the third layer 215 may have a third edge 1115. In some examples, the first edge 1105, the second edge 1110, and the third edge 1115 may have substantially the same shape so that adjacent faces of the first layer 205 and the second layer 210 and adjacent faces of the second layer 210 and the third layer 215 are geometrically similar. The first edge 1105, the second edge 1110, and the third edge 1115 may also be congruent in some examples, so that adjacent faces of the first layer 205 and the second layer 210 and adjacent faces of the second layer 210 and the third layer 215 are substantially coextensive and have substantially the same surface area. In the example of FIG. 11, the first edge 1105 defines a larger face of the first layer 205 than the faces of the second layer 210 and third layer 215 defined by the second edge 1110 and the third edge 1115, respectively, and the larger face of the first layer 205 extends past the smaller faces of the second edge 1110 and the third edge 1115. Additionally, in the example of FIG. 11, the second edge 1110 defines a larger face of the second layer 210 than the face of the third layer 215 defined by the third edge 1115.

The faces defined by the first edge 1105, the second edge 1110, and/or the third edge 1115 may also be geometrically similar to the treatment aperture 915 in some embodiments, as illustrated in the example of FIG. 11, and may be larger than the treatment aperture 915. The fourth layer 905 may have an overlay margin 1120 around the treatment aperture 915, which may have an additional adhesive disposed therein. As illustrated in the example of FIG. 11, the treatment aperture 915 may be an ellipse or a stadium in some embodiments. The treatment aperture 915 may have an area that is equal to about 20% to about 80% of the area of the fourth layer 905 in some examples. The treatment aperture 915 may also have an area that is equal to about 20% to about 80% of the area of a face of defined by the first edge 1105 of the first layer 205. A width in a range of about 90 millimeters to about 110 millimeters and a length in a range of about 150 millimeters to about 160 millimeters may be suitable for some embodiments of the treatment aperture 915. For example, the width of the treatment aperture 915 may be about 100 millimeters, and the length may be about 155 millimeters. In some embodiments, a suitable width for the overlay margin 1120 may be about 2 millimeters to about 3 millimeters. For example, the overlay margin 1120 may be coextensive with an area defined between the treatment aperture 915 and the first edge 1105, and the adhesive may secure the first layer 205, the second layer 210, and/or the third layer 215 to the fourth layer 905.

In other embodiments, for example, the faces defined by the first edge 1105, the second edge 1110, and/or the third edge 1115 may also be geometrically similar to the treatment aperture 915 and may be the same size as the treatment aperture 915. Thus, the treatment aperture 915 may also have an area that is equal to the area of a face of defined by the first edge 1105 of the first layer 205.

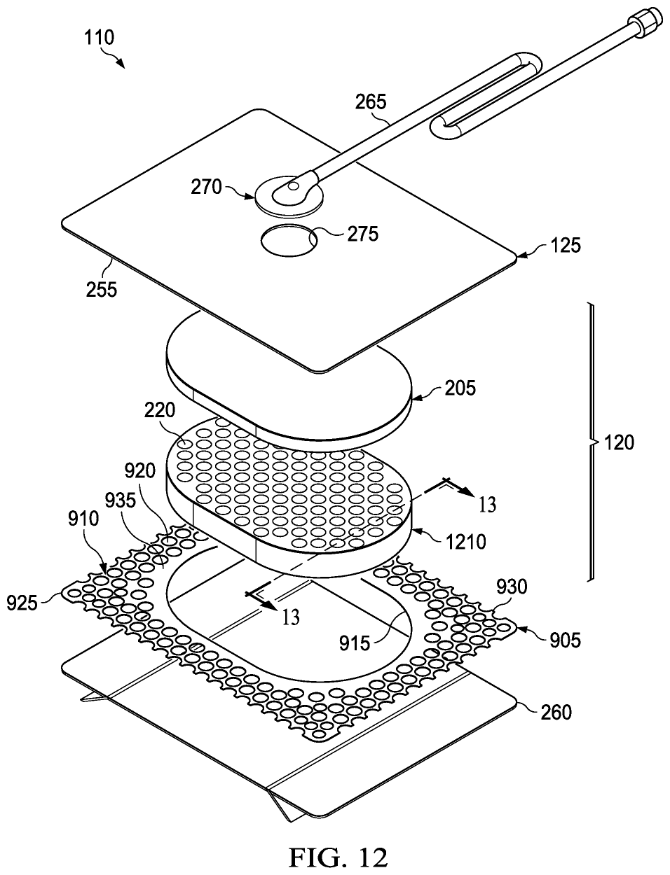

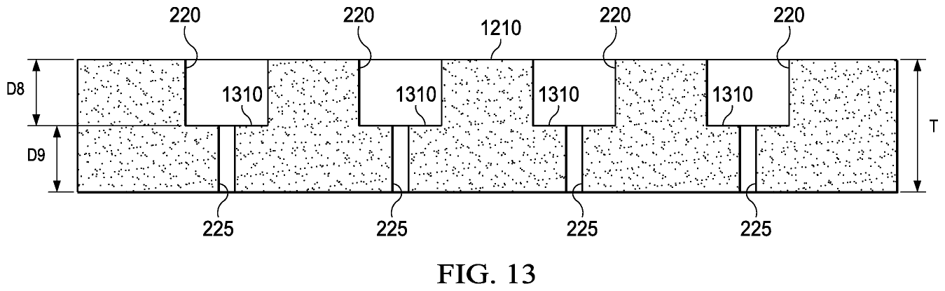

FIG. 12 and FIG. 13 illustrate another example of the dressing 110 having a second layer 1210 that may combine certain features of the second layer 210 and the third layer 215. In some embodiments, the second layer 1210 may comprise or consist of a material that is substantially similar or identical to the second layer 210. For example, the second layer 1210 may comprise or consist essentially of a closed-cell foam. The second layer 1210 of FIG. 12 and FIG. 13 generally has a first planar surface and a second planar surface opposite the first planar surface. The thickness T of the second layer 1210 between the first planar surface and the second planar surface may also vary according to needs of a prescribed therapy. In some embodiments, the second layer 1210 may have a thickness T in a range of about 0.5 millimeters to about 20 millimeters. In some embodiments, the second layer 1210 may have a thickness T in a range of about 2 millimeter to about 6 millimeters. In some embodiments, for example, the thickness may be less than 0.5 millimeters. In some embodiments, for example, the thickness may be greater than 20 millimeters. As with the second layer 210 and the third layer 215, the second layer 1210 may be hydrophobic. In some embodiments, the second layer 1210 may have varying degrees of hydrophobicity throughout the thickness T. That is, for example, in some embodiments, the second layer 1210 may be more hydrophobic at the second planar surface and may become less hydrophobic toward the first planar surface. This varying degree of hydrophobicity may assist in removing exudate from the tissue site and directing it to the first layer 205.

As illustrated in FIG. 12 and FIG. 13, the second layer 210 may have one or more apertures 220 and one or more fluid restrictions 225 fluidly coupled with the one or more apertures 220. As further illustrated in FIG. 13, the apertures 220 extend from the first planar surface to a depth D8 into the second layer 1210 terminating in a floor 1310, and the fluid restrictions 225 extend a depth D9 from the floor 1310 to the second planar surface of the second layer 1210. The sum of depth D8 and depth D9 may be equal to the thickness T of the second layer 1210. Each aperture 220 is in fluid communication with a corresponding fluid restriction 225.

Individual components of the dressing 110 may be bonded or otherwise secured to one another with a solvent or non-solvent adhesive, or with thermal welding, for example, without adversely affecting fluid management. Further, the first layer 205, second layer 210, and/or the third layer 215 may be coupled to the interior border 935 or the overlay margin 1120 of the fourth layer 905 in any suitable manner, such as with a weld or an adhesive, for example.

Although dressings 110 are described herein having three and four layers, it will be understood that additional layers may be included without departing from the scope of the disclosure. Additionally, in some embodiments the layers may be of different colors. That is, for example, the first layer 205 may be a first color, the second layer 210 may be a second color, the third layer 215 may be a third color, the four layer 905 may be a fourth color, and the second layer 1210 may be a fifth color, wherein each of the first, second, third, fourth, and fifth colors are different. In other embodiments, for example, one or more of the first layer 205, the second layer 210, the third layer 215, the fourth layer 905, and the second layer 1210 may be transparent, translucent, or opaque.

The cover 125, the first layer 205, the second layer 210, the third layer 215, the fourth layer 905, the second layer 1210, or various combinations may be assembled before application or in situ. For example, the first layer 205 may be coupled to the second layer 210, and the second layer 210 may be coupled to the third layer 215 in some embodiments. The cover 125 may be disposed over the first layer 205 and coupled to the fourth layer 905 around the first layer 205 in some embodiments. In some embodiments, one or more layers of the tissue interface 120 may be coextensive. For example, the second layer 210 may be cut flush with the edge of the first layer 205, and the third layer 215 may be cut flush with the edge of the second layer 210. In some embodiments, the dressing 110 may be provided as a single, composite dressing. For example, the fourth layer 905 may be coupled to the cover 125 to enclose the first layer 205, the second layer 210, and the third layer 215, wherein the fourth layer 905 may be configured to face a tissue site. As shown in FIG. 2, for example, the dressing 110 may not include the fourth layer 905. Accordingly, the third layer 215 may be configured to face a tissue site.

In use, the release liner 260 (if included) may be removed to expose the fourth layer 905 (if included) and/or the cover 125, which can provide a lower surface of the dressing 110 to be placed within, over, on, or otherwise proximate to a tissue site, particularly a surface tissue site and adjacent epidermis. The second layer 210, the third layer 215, and the fourth layer 905 (if included) may be interposed between the first layer 205 and the tissue site, which can substantially reduce or eliminate adverse interaction between the first layer 205 and the tissue site. For example, the fourth layer 905 may be placed over a surface wound (including edges of the wound) and undamaged epidermis to prevent direct contact with the first layer 205. In some applications, the treatment aperture 915 of the fourth layer 905 may be positioned adjacent to, proximate to, or covering a tissue site. In some applications, at least some portion of the third layer 215 and the fluid restrictions 225 may be exposed to a tissue site through the treatment aperture 915. The periphery 910 of the fourth layer 905 may be positioned adjacent to or proximate to tissue around or surrounding the tissue site. The fourth layer 905 may be sufficiently tacky to hold the dressing 110 in position, while also allowing the dressing 110 to be removed or re-positioned without trauma to the tissue site.

Removing the release liner 260 can also expose the adhesive 255, and the cover 125 may be attached to an attachment surface, such as the periphery 910 or other area around the treatment aperture 915 and the first layer 205. The adhesive 255 may also be attached to epidermis peripheral to a tissue site, around the first layer 205 and the second layer 210. For example, the adhesive 255 may be in fluid communication with an attachment surface through the apertures 920 in at least the periphery 910 of the fourth layer 905. The adhesive 255 may also be in fluid communication with the edges 930 through the apertures 920 exposed at the edges 930.

Once the dressing 110 is in the desired position, the adhesive 255 may be pressed through the apertures 920 to bond the dressing 110 to the attachment surface. The apertures 920 at the edges 930 may permit the adhesive 255 to flow around the edges 930 for enhancing the adhesion of the edges 930 to an attachment surface.

In some embodiments, the apertures 920 may be sized to control the amount of the adhesive 255 exposed through the apertures 920. For a given geometry of the corners 925, the relative sizes of the apertures 920 may be configured to maximize the surface area of the adhesive 255 exposed and in fluid communication through the apertures 920 at the corners 925. For example, the edges 930 may intersect at substantially a right angle, or about 90 degrees, to define the corners 925. In some embodiments, the corners 925 may have a radius of about 10 millimeters. Further, in some embodiments, three of the apertures 920 may be positioned in a triangular configuration at the corners 925 to maximize the exposed surface area for the adhesive 255. In other embodiments, the size and number of the apertures 920 in the corners 925 may be adjusted as necessary, depending on the chosen geometry of the corners 925, to maximize the exposed surface area of the adhesive 255. Further, the apertures 920 at the corners 925 may be fully contained within the fourth layer 905, substantially precluding fluid communication in a lateral direction exterior to the corners 925. The apertures 920 at the corners 925 being fully contained within the fourth layer 905 may substantially preclude fluid communication of the adhesive 255 exterior to the corners 925, and may provide improved handling of the dressing 110 during deployment at a tissue site. Further, the exterior of the corners 925 being substantially free of the adhesive 255 may increase the flexibility of the corners 925 to enhance comfort.

In some embodiments, the bond strength of the adhesive 255 may vary based on the configuration of the fourth layer 905. For example, the bond strength may vary based on the size of the apertures 920. In some examples, the bond strength may be inversely proportional to the size of the apertures 920. Additionally or alternatively, the bond strength may vary in different locations, for example, if the size of the apertures 920 varies. For example, a lower bond strength in combination with larger apertures 920 may provide a bond comparable to a higher bond strength in locations having smaller apertures 920.

The geometry and dimensions of the tissue interface 120, the cover 125, or both may vary to suit a particular application or anatomy. For example, the geometry or dimensions of the tissue interface 120 and the cover 125 may be adapted to provide an effective and reliable seal against challenging anatomical surfaces, such as an elbow or heel, at and around a tissue site. Additionally or alternatively, the dimensions may be modified to increase the surface area for the third layer 215 to enhance the movement and proliferation of epithelial cells at a tissue site and reduce the likelihood of granulation tissue in-growth.

Thus, the dressing 110 can provide a sealed therapeutic environment proximate to a tissue site, substantially isolated from the external environment, and the negative-pressure source 105 can reduce the pressure in the sealed therapeutic environment. The treatment aperture 915 can provide an open area for delivery of negative pressure and passage of wound fluid through the third layer 215, the second layer 210, and the first layer 205. The fourth layer 905 may provide an effective and reliable seal against challenging anatomical surfaces, such as an elbow or heel, at and around a tissue site. Further, the dressing 110 may permit re-application or re-positioning, to correct air leaks caused by creases and other discontinuities in the dressing 110, for example. The ability to rectify leaks may increase the efficacy of the therapy and reduce power consumption in some embodiments.

If not already configured, the dressing interface 270 may be disposed over the aperture 275 and attached to the cover 125. The fluid conductor 265 may be fluidly coupled to the dressing interface 270 and to the negative-pressure source 105.

Negative pressure applied through the tissue interface 120 can create a negative pressure differential across the fluid restrictions 225 in the third layer 215, which can open or expand the fluid restrictions 225 and can draw a portion of the third layer 215 adjacent to the fluid restrictions 225 into the apertures 220 in the second layer 210. For example, in some embodiments in which the fluid restrictions 225 may comprise substantially closed fenestrations through the third layer 215, a pressure gradient across the fenestrations can strain the adjacent material of the third layer 215 and increase the dimensions of the fenestrations to allow liquid movement through them, similar to the operation of a duckbill valve. Opening the fluid restrictions 225 can allow exudate and other liquid movement through the fluid restrictions 225, into the apertures 220 of the second layer 210 and then into the first layer 205. The first layer 205 can provide passage of negative pressure and wound fluid, which can be collected in the container 115. Changes in pressure can also cause the first layer 205 to expand and contract, and the second layer 210, the third layer 215, and/or the fourth layer 905 (if included) may protect the epidermis from irritation that could be caused by expansion, contraction, or other movement of the first layer 205. For example, in some embodiments, the overlay margin 1120 may be disposed between the first layer 205 and epidermis around a tissue site. The second layer 210, the third layer 215, and the fourth layer 905 can also substantially reduce or prevent exposure of a tissue site to the first layer 205, which can inhibit growth of tissue into the first layer 205. For example, the third layer 215 may cover the treatment aperture 230 to prevent direct contact between the first layer 205 and a tissue site.

If the negative-pressure source 105 is removed or turned off, the pressure differential across the fluid restrictions 225 can dissipate, allowing the fluid restrictions 225 to close and prevent exudate or other liquid from returning to the tissue site through the third layer 215.

In some applications, a filler may also be disposed between a tissue site and the third layer 215 and/or fourth layer 905 (if included). For example, if the tissue site is a surface wound, a wound filler may be applied interior to the periwound, and the fourth layer 905 may be disposed over the periwound and the wound filler. In some embodiments, the filler may be a manifold, such as an open-cell foam. The filler may comprise or consist essentially of the same material as the first layer 205 in some embodiments.

Additionally or alternatively, instillation solution or other fluid may be distributed to the dressing 110, which can increase the pressure in the tissue interface 120. The increased pressure in the tissue interface 120 can create a positive pressure differential across the fluid restrictions 225 in the third layer 215, which can open the fluid restrictions 225 to allow the instillation solution or other fluid to be distributed to the tissue site.

The systems, apparatuses, and methods described herein may provide significant advantages. For example, the manufacturing process of constructing the second layer 210 and the third layer 215 out of closed-cell foam and forming the fluid restrictions 225 in the third layer 215 can reduce complexity and cost of manufacturing. This is because the apertures 220 can be formed in the second layer 210, the third layer 215 can be coupled to the second layer 210, and then the apertures 220 can be used as a guide for forming the fluid restrictions 225 in the third layer 215.

The depth of the apertures 220 can provide more space for the fluid restrictions 225 to move and dynamically open. That is, the fluid restrictions 225 and the third layer 215 adjacent to the fluid restrictions 225 can deform inward into the apertures 220 under negative pressure. Moreover, locating the fluid restrictions 225 between the tissue site and the apertures 220 can reduce, prevent or eliminate tissue in-growth into the apertures 220.

Constructing the third layer 215 and the second layer 210 from hydrophobic materials can also provide additional benefits. For example, hydrophobic properties of the third layer 215 can prevent exudate from causing the third layer 215 to swell and can prevent opening of the fluid restrictions 225 in the absence of negative pressure. Additionally, in some embodiments, constructing the second layer 210 from a hydrophobic material that is less hydrophobic than the material of the third layer 215 can allow the second layer 210 to wick exudate away from the third layer 215 and direct it to the first layer 205. A closed-cell foam construction of the second layer 210 and the third layer 215 can also substantially reduce or prevent tissue in-growth into the second layer 210 and the third layer 215. Accordingly, the characteristics and orientation of the stack formed by some embodiments of the first layer 205, the second layer 210, and the third layer 215 can improve fluid removal from the tissue site while reducing, preventing or eliminating tissue in-growth. That is, the systems, apparatuses, and methods described herein can provide a contact surface and manifolding media that is highly resistant to tissue in-growth.

The systems, apparatuses, and methods described herein may provide yet additional significant advantages. For example, the dressing 110 can be simple to apply, reducing the time to apply and remove. In some embodiments, for example, the dressing 110 may be a fully-integrated negative-pressure therapy dressing that can be applied to a tissue site (including on the periwound) in one step, without being cut to size, while still providing or improving many benefits of other negative-pressure therapy dressings that require sizing. Such benefits may include good manifolding, beneficial granulation, protection of the peripheral tissue from maceration, protection of the tissue site from shedding materials, and a low-trauma and high-seal bond. These characteristics may be particularly advantageous for surface wounds having moderate depth and medium-to-high levels of exudate. Some embodiments of the dressing 110 may remain on the tissue site for at least 5 days, and some embodiments may remain for at least 7 days. Antimicrobial agents in the dressing 110 may extend the usable life of the dressing 110 by reducing or eliminating infection risks that may be associated with extended use, particularly use with infected or highly exuding wounds.

While shown in a few illustrative embodiments, a person having ordinary skill in the art will recognize that the systems, apparatuses, and methods described herein are susceptible to various changes and modifications that fall within the scope of the appended claims. Moreover, descriptions of various alternatives using terms such as “or” do not require mutual exclusivity unless clearly required by the context, and the indefinite articles “a” or “an” do not limit the subject to a single instance unless clearly required by the context. Components may be also be combined or eliminated in various configurations for purposes of sale, manufacture, assembly, or use. For example, in some configurations the dressing 110, the container 115, or both may be eliminated or separated from other components for manufacture or sale. In other example configurations, the controller 130 may also be manufactured, configured, assembled, or sold independently of other components.

The appended claims set forth novel and inventive aspects of the subject matter described above, but the claims may also encompass additional subject matter not specifically recited in detail. For example, certain features, elements, or aspects may be omitted from the claims if not necessary to distinguish the novel and inventive features from what is already known to a person having ordinary skill in the art. Features, elements, and aspects described in the context of some embodiments may also be omitted, combined, or replaced by alternative features serving the same, equivalent, or similar purpose without departing from the scope of the invention defined by the appended claims.

1. A dressing for treating a tissue site with negative pressure, the dressing comprising:
a first layer comprising a manifold;
a second layer coupled to the first layer, the second layer comprising a first closed-cell foam;
a plurality of apertures through the first closed-cell foam;
a third layer coupled to the second layer opposite the first layer, comprising a second closed-cell foam; and
a plurality of fluid restrictions through the second closed-cell foam that are configured to expand in response to a pressure gradient across the second closed-cell foam, the plurality of fluid restrictions fluidly coupled with at least some of the plurality of apertures in the second layer.

2. The dressing of claim 1, wherein the third layer is hydrophobic.

3. The dressing of claim 1, wherein one or more of the first closed-cell foam and the second closed-cell foam is silicone, polyurethane, or ethylene vinyl acetate.

4.-5. (canceled)

6. The dressing of claim 1, wherein the plurality of fluid restrictions in the third layer are aligned with at least some of the plurality of apertures through the second layer.

7. The dressing of claim 1, wherein one or more of the second layer and the third layer has a thickness in a range of 1 millimeter to 3 millimeters.

8. The dressing of claim 1, wherein one or more of the second layer and the third layer has a pore size in a range of 0.2 millimeters to 1 millimeter.

9. The dressing of claim 1, wherein one or more of the second layer and the third layer has a hardness of about 10 Shore A to about 50 Shore A.

10. The dressing of claim 1, wherein the third layer comprises an exposed surface that is smooth.

11. The dressing of claim 1, wherein the fluid restrictions comprise a plurality of slots configured to permit fluid flow and inhibit exposure of the first layer to the tissue site.

12.-15. (canceled)

16. The dressing of claim 1, wherein the fluid restrictions comprise a plurality of slots, each of the slots having a length less than 5 millimeters and a width less than 2 millimeters.

17.-29. (canceled)

30. The dressing of claim 1, wherein the fluid restrictions are distributed across the third layer in a uniform pattern.

31.-33. (canceled)

34. The dressing of claim 1, wherein the second layer is hydrophobic.

35. The dressing of claim 34, wherein the second layer is less hydrophobic than the third layer.

36.-44. (canceled)

45. The dressing of claim 1, wherein the apertures comprise a plurality of holes, each of the holes having a diameter of less than 5 millimeters.

46.-58. (canceled)

59. The dressing of claim 1, wherein at least one of the apertures is sized to permit deflection of the third layer proximate the plurality of fluid restrictions of about 1 millimeter into and out of the at least one aperture.

60. The dressing of claim 1, wherein the manifold comprises a porous foam.

61.-80. (canceled)

81. The dressing of claim 1, further comprising a cover coupled to the first layer opposite the second layer.

82.-84. (canceled)

85. The dressing of claim 81, further comprising a fourth layer coupled to the cover, the fourth layer comprising a sealing layer having a treatment aperture and a plurality of perforations around the treatment aperture.

86.-103. (canceled)

104. A dressing for treating a tissue site with negative pressure, the dressing comprising:
a cover;
a manifold;
a support layer comprising a first closed-cell foam having a plurality of apertures; and
a fluid control layer comprising a second closed-cell foam having a plurality of perforations fluidly coupled with the plurality of apertures;
wherein the cover, the manifold, the support layer, and the fluid control layer are assembled in a stacked relationship, and the fluid control layer is configured to contact the tissue site.

105. A dressing for treating a tissue site with negative pressure, the dressing comprising:
a cover;
a gel layer coupled to the cover, the gel layer comprising an open central window and a plurality of openings around the open central window;
a manifold;
a support layer comprising a first closed-cell foam having a plurality of apertures; and
a fluid control layer comprising a second closed-cell foam having a plurality of perforations fluidly coupled with the plurality of apertures;
wherein the cover, the gel layer, the manifold, the support layer, and the fluid control layer are assembled in a stacked relationship, and the fluid control layer is configured to contact the tissue site.

106.-109. (canceled)
